(19) United States
(12) Patent Application Publication
(10) Pub. No.: US 2025/0366971 A1
(43) Pub. Date: Dec. 4, 2025

(54) MESH EXPANSION SLEEVE

(71) Applicant: Board of Supervisors of Louisiana State University and Agricultural and Mechanical College, Baton Rouge, LA (US)

(72) Inventors: Jonathan Steven ALEXANDER, Shreveport, LA (US); Giovanni Francesco SOLITRO, Shreveport, LA (US); Donald SORRELLS, Shreveport, LA (US)

(21) Appl. No.: 19/236,666

(22) Filed: Jun. 12, 2025

Related U.S. Application Data
(63) Continuation-in-part of application No. 17/901,847, filed on Sep. 1, 2022.
(60) Provisional application No. 63/217,437, filed on Jul. 1, 2021.
(60) Provisional application No. 63/659,190, filed on Jun. 12, 2024.

(51) Int. Cl. A61F 2/04; A61L 27/54

(52) U.S. Cl. CPC A61F 2/04; A61L 27/54; A61F 2002/045; A61F 2210/0071; A61F 2220/0008; A61F 2230/0069; A61F 2230/0091; A61F 2250/0067; A61L 2300/43; A61L 2430/22

(57) ABSTRACT

A device is described herein including an expansion sleeve that comprises a cylindrical woven material, wherein the cylindrical woven material comprises helical trusses, wherein an outer surface of the cylindrical woven material is coated with a biologically active material.

### RELATED APPLICATIONS

This application is a continuation in part application of U.S. application Ser. No. 17/901,847, filed Sep. 1, 2022, which claims the benefit of Unites States Application No. 63/217,437, filed Jul. 1, 2021.

This application claims the benefit of U.S. Application No. 63/659,190, filed Jun. 12, 2024.

### TECHNICAL FIELD

The disclosure herein involves a medical device for lengthening tissue structures.

### BACKGROUND

Pediatric surgeons often encounter patients with intestinal failure due to inadequate intestinal length (short gut syndrome/SGS). These patients are challenging because gut adaptation and enteral absorption remains low. Patients with short gut compensate with gastroparesis and slow dilation of the intestinal diameter. This process may take weeks, months or years requiring supplemental parenteral nutrition for adequate growth. Native elongation of the short intestine is frequently limited. Two surgical elongation procedures have been developed—the Bianchi and serial transverse enteroplasty (STEP) procedure. Both procedures require that the patient has developed sufficient dilation of bowel diameter. The medical care of these patients relies on daily total parenteral nutrition (TPN), central line access, and meticulous electrolyte management.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a Roux-en-Y surgical model to promote gut elongation, under an embodiment.

FIGS. 2A-2D shows histology of the small intestine (left) with IES. The histology shows significant wall thinning in all layers of the small and large intestines in response to dilation of the lumens with IES, under an embodiment.

FIG. 3 shows gut expansion data with respect to small and large intestine, under an embodiment.

FIGS. 4A-4I show deployment of mesh expansion sleeve, under an embodiment.

FIG. 5 shows mechanical characterization of the DE device during the expansion phase at strains of 50% (FIG. 5A), 25% (FIG. 5B), and at its nominal length (FIG. 5C), under an embodiment.

FIG. 6 shows a representative sleeve and introduction into intestine (left) and intestine after expansion of the sleeve (right), under an embodiment.

FIG. 7 shows mechanical testing of IES bowel connection, IES device at top (blue) is sutured to intestinal wall (lower section) and load failure performance analyzed, under an embodiment.

FIG. 8 shows sleeve placement in the rabbit small intestine versus length of IES (n=3 for each group), under an embodiment.

FIG. 9 shows sleeve placement in the rabbit colon versus length of IES (n=three for each group), under an embodiment.

FIG. 10 shows a mesh expansion sleeve, under an embodiment.

FIG. 11 shows a mesh expansion sleeve, under an embodiment.

FIG. 12 shows proximal limb, distal limb and Roux-en-Y configuration, under an embodiment.

FIG. 13 shows histology results, under an embodiment.

FIG. 14 shows pre Roux length and post Roux length, under an embodiment.

FIG. 15 shows Mayer-Rokitansky-Küster-Hauser (MRKH) Syndrome, under an embodiment.

FIGS. 16-19 show conventional treatments for MRKH Syndrome, under an embodiment.

FIG. 20A shows a vaginal expansion sleeve (VES), under an embodiment.

FIG. 20B shows biomechanical characterization testing of a VES design, under an embodiment.

FIG. 20C shows VES post-insertion into the rat vaginal canal (left) and after suturing into place (right), under an embodiment.

FIG. 21 shows a mechanical characterization of a VES design, under an embodiment.

FIG. 22 shows a harvested vaginal canal with the inserted VES, under an embodiment.

FIG. 23 shows histological data, under an embodiment. Histology demonstrated significant thinning across the epithelial, muscular, and serosal layers when compared to the control vaginal tissue samples, resulting in total vaginal wall thinning.

FIG. 24 shows histological data of muscle fibers in control and stretched vaginal wall tissue, under an embodiment.

FIG. 25 shows a schematic view of an expansion sleeve, under an embodiment.

FIG. 26 shows components of an expansion sleeve, under an embodiment.

FIG. 27 shows an expansion sleeve, under an embodiment.

FIG. 28 shows an expansion sleeve, under an embodiment.

FIG. 29A shows precontracted expansion sleeves over a class pipette for administering the PVA coating, under an embodiment.

FIG. 29B shows precontracted expansion sleeves in a desiccator crosslinking the PVA coating with glutaraldehyde (GA) and sulfuric acid, under an embodiment.

FIG. 29C shows precontracted expansion sleeves in a desiccator crosslinking the PVA coating with glutaraldehyde (GA) and sulfuric acid, under an embodiment.

FIG. 29D shows a concentration grid of GLP-2 solution (in ug) and respective expansion sleeves, under an embodiment.

FIG. 29E shows a 24-well plate containing a 2-component lipid substrate system that develops a blue reaction product when reacted with phosphatase-labeled conjugates indicating GLP-2 concentration on the PVA coated sleeves, under an embodiment.

FIG. 30 shows a standard curve at 620 nm absorbance and concentrations of GLP-2 calculated under said curve, under an embodiment.

FIG. 31 shows a bar graph of GLP-2 concentration (ug/cm²) for standard solutions (250 ug, 100 μg, 50 ug, 25 ug, and 0 ug), IES solutions, and VES solutions, under an embodiment.

FIG. 32 shows precontraction and compression of an IES device, under an embodiment.

FIG. 33 shows creation of the Roux-En-Y, under an embodiment.

FIG. 34 shows Intestinal Length Pre and Post Expansion, under an embodiment.

### DETAILED DESCRIPTION

### Example 1

Pediatric surgeons often encounter patients with intestinal failure due to inadequate intestinal length (short gut syndrome/SGS). These patients are challenging because gut adaptation and enteral absorption remains low. Patients with short gut compensate with gastroparesis and slow dilation of the intestinal diameter.¹ This process may take weeks, months or years requiring supplemental parenteral nutrition for adequate growth.² Native elongation of the short intestine is frequently limited. Two surgical elongation procedures have been developed—the Bianchi³ and serial transverse enteroplasty (STEP) procedure⁴. Both procedures require that the patient has developed sufficient dilation of bowel diameter.⁵ The medical care of these patients relies on daily total parenteral nutrition (TPN), central line access, and meticulous electrolyte management.

‘Distraction enterogenesis (DE), or gut wall remodeling/stretching using various devices has been shown to elongate the intestine in several animal models.⁷ These devices promote longitudinal tension within a segment of intestine resulting in elongation of the segment by 50% or more.⁸ Some of these devices include springs,⁹ and balloon devices¹⁰. Initial studies have placed these devices out of continuity of the gastrointestinal tract such as in a ‘Roux limb’.¹¹ Placement in a Roux limb initially is theoretically safer than placing in continuity. Spring devices have been successfully placed in continuity in a rat model.¹² The potential for improvement in the clinical course of these patients is tremendous with decreased days on TPN, decreased need for central access and decreased line infections.

There are several potential benefits/advantages of our lab's intestinal expansion sleeves (IES) over other prototypes. Unlike coils, these devices can accommodate significant amounts of growth factors which can maintain gut wall integrity which increases success of implanted devices. In our model¹² (Clayton et al., 2021) The contracted device expands radially in proportion to the amount of shortening in length of the IES. The radial expansion should make the device easier to secure to the intestine in a future noninvasive model. Lastly, the fully expanded sleeve has a decreased diameter making eventual passage of the IES in the stool more likely.

Our DE device is an implantable, hollow porous mesh sleeve intended to be attached at each end to the intestine with absorbable sutures. The device is designed to exert a linear force curve as it expands lengthening the intestine. The segment would eventually be passed in stool once the absorbable sutures have dissolved. To save the patient operations and potential intestinal length, the device could be fixed to the end of a long transpyloric feeding tube and deployed without surgery. Multiple deployments of these sleeves should be possible.

Short gut syndrome (SGS) is a clinical phenomenon affecting about 30,000 individuals each year where gut length is significantly reduced, compromising the ability to sustain adequate gastrointestinal nutrient absorption. This syndrome usually reflects the catastrophic loss of intestinal tissue from a volvulus (twisting and ischemia of the intestine), infection (necrotizing enterocolitis), immune disorders (Crohn's disease), trauma and idiopathic causes. The small intestine absorbs most nutrients and adapts to tissue loss by dilating in diameter which increases surface area for absorption but not length. Children and adults require years to become independent of IV nutrition. Sometimes SGS-afflicted individuals never become fully independent of IV nutrition/total parenteral nutrition (TPN). The cost for IV nutrition and/or hospitalization to treat line infections is substantial. In the long term, SGS patients may develop multiple central line infections, liver disease and organ failure.

The Medicare cost per short gut patient receiving TPN varies between $64,000 to $128,000 annually. For the 30,000 US patients receiving TPN, this amounts to societal cost of $1.9 billion (about $6 per person in the US) annually. This figure only includes hyperalimentation solution and does not consider costs for hospitalizations, labs, and surgeries. These patients are frequently impaired from fully contributing to society through a vocation of their choice. This cost to society is difficult to measure but present.

Intestinal expansion sleeves (IES) have tremendous potential to increase the length of residual intestine in such patients. With longer intestinal length, these patients should gain independence from TPN, require less hospitalizations and decrease need for central venous lines with significant improvement in quality-of-life scores. Distraction enterogenesis has the potential to treat short bowel syndrome. Our IES offers multiple improvements compared to prior devices. The sleeve is porous and should be deployable in continuity with GI tract. Also, the IES will not require removal as it will be held in place with absorbable sutures. The device has limited axial expansion and excellent linear expansion as shown by this study. Lastly, the future use of IES may enable device to be deployed without surgery over a feeding catheter as well as coated with drugs to promote healing or decrease inflammation.

### Competition and Advantage

Currently there are no IES devices in testing on humans. Distraction enterogenesis is a new field of study and we have published a unique methodology for achieving IES distraction (gut lengthening, Sorrells et al., 2021). Only one group, Dunn et al. has employed metal springs to achieve bowel lengthening like our device. There are several critical advantages to our prototype over the typical spring. First, IES sleeve diameters diminish as gut lengthening is achieved. This allows for spontaneous and easy passage and elimination of the device through the GI tract after elongation. Second the sleeve material can be impregnated with materials which can enhance the process of gut lengthening. IES sleeves have tremendous surface area to bind drugs compared to metal springs. Third, as the device is contracted, the diameter enlarges slightly. This is a potential property of the sleeve which may enable a future nonsurgical method of deployment of the sleeve into the intestine.

### Ease/Cost of Proof-of-Concept

Using our approach, we can gain significant increases in gut length up to 30%. Mechanical testing performed in our lab also determined the optimal number of sutures to secure the device. (These data were presented at the European Colorectal Club meeting in Athens 2021 and published in the journal Pediatric Surgery International). Our lab has recently continued to evaluate this model with testing in an ex vivo rat model in preparation for surgical studies in vivo. These hollow, tubular IES sleeves are 30 mm in length and compress to approximately 20 mm. Therefore, our IES sleeve has the potential to gain 50% increase in gut length over time. This approach would allow us to implant expansion sleeves in a Roux limb of intestine on the rat in vivo. The Roux-en-Y procedure will isolate the sleeve in a limb of intestine which is unlikely to result in iatrogenic complications such as peritonitis from erosion, bowel obstruction, or fistulae. Once we have proven that our IES sleeve creates length while maintaining gut structure, we would place sleeves ‘in continuity’ (Aim 3 below). Rats will be kept with IES sleeves in place for 4 weeks. At sacrifice, tissue will be analyzed for tissue histology, biomechanical properties, motility, and tissue markers. These experiments will be repeated using IES which are infused with GLP-2, a hormone which has been used to promote gut healing and absorptive capacity.

### R&D Plan/Milestones

Aim 1—Extend functional gut length by surgically placing expansion sleeves into Roux-en-Y. Hypothesis: IES sleeves are implanted in a Roux-en-Y model to increase intestinal length as compared to controls. Eighteen rats undergo laparotomy with creation of a Roux-en-Y as described by Dubrovsky et al., 2019¹³. In the Roux limb, the intestinal expansion device will be placed. Four of these rats will serve as controls with Roux-en-Y created but no sleeve placed. The remaining fourteen rats will have a sleeve placed in the Roux limb. The sleeve will be secured in place with nonabsorbable sutures. The sleeves are radiopaque, and the rats will have x-rays taken at 3 intervals during the 4 weeks. After 4 weeks, rats will be sacrificed and bowel length measured, histology and markers. Toleration of diet postoperatively, weight gain, gut wall dilation and absence of intestinal obstruction will be studies. Segments of intestine with IES will be evaluated for increases in length compared to segments containing control IES.

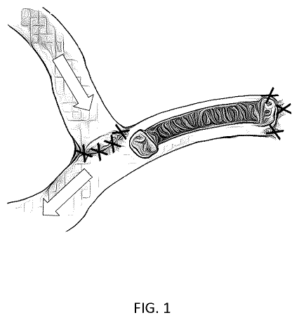

FIG. 1 shows a Roux-en-Y surgical model to promote gut elongation, under an embodiment. The same type of tissue extender would be placed and/or sutured into the blind limb, except that in Aim 2, tissue extenders would contain GLP2 to enhance gut restitution. Arrows show chyle flow outside of the blind limb.

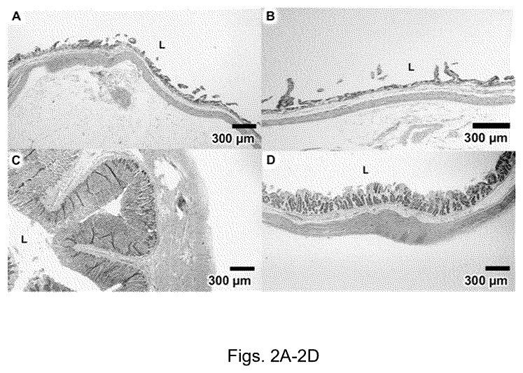

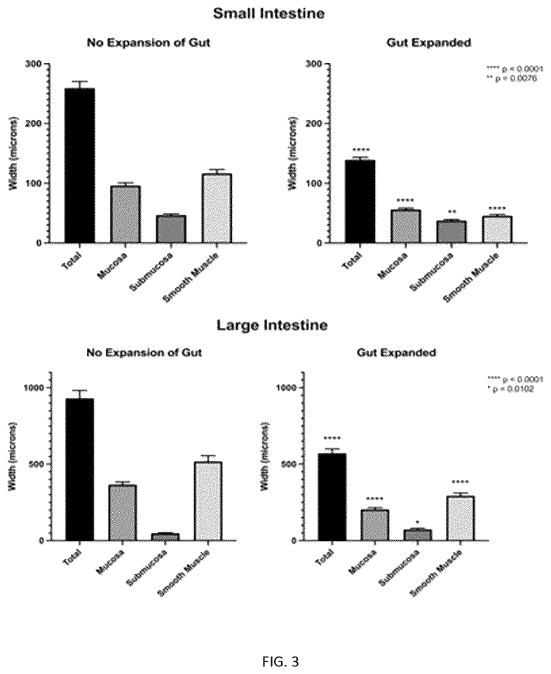

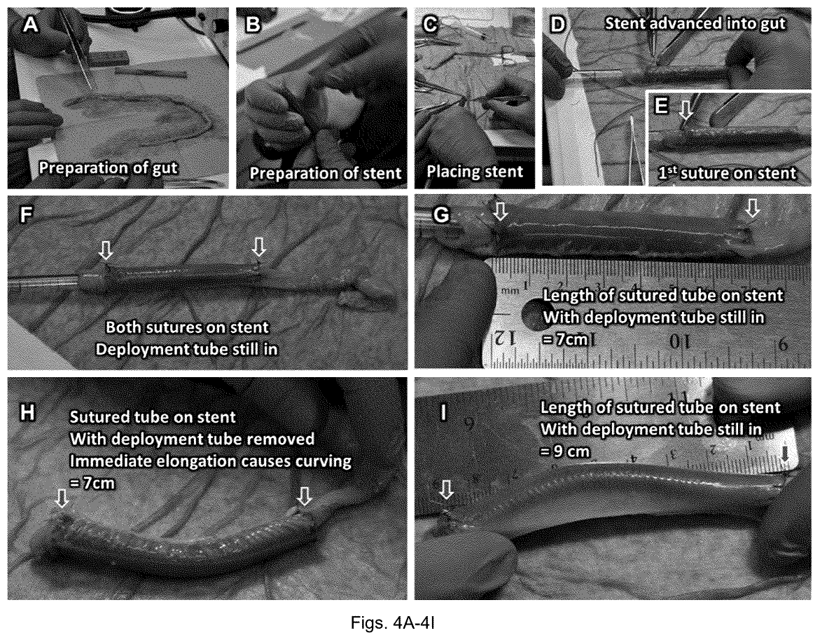

Aim 2—Gattex (teduglutide) impregnated sleeves will develop more extensive gut surface to promote gut absorption. Hypothesis: We have observed that gut elongation by stretching is associated with thinning of several layers of the intestinal wall. As seen in FIGS. 2A-2D histology of the small intestine (left) with IES showed significant wall thinning in all layers of the small and large intestines in response to dilation of the lumens with IES. A) Small intestine undilated and B) dilated; C) Large intestine undilated and D) dilated. Bar=300 m. FIG. 3 shows comparisons made by t-test between non-expanded and expanded states for total thickness and each tissue layer. From Clayton et al., 2021.

To enhance gut wall thickening during DE, we will impregnate IES with covalently attached GLP2, a gut trophic factor which improves gut wall thickening¹⁴. GLP2 impregnated sleeves should therefore promote increased gut length as well hypertrophy of the mucosal area which is critical to nutritional absorption. Gattex is a GLP2 analog which has been approved by the FDA for use in humans with short bowel syndrome. Patients receive this drug as a subcutaneous injection of 0.03-0.15 mg per KG daily. The GLP2 analog stimulates mucosal growth. Impregnating an intestinal expansion sleeve with this drug may have an additive effect in gut expansion. We will covalently activate IES sleeve by covalently binding GLP2 to polyvinyl alcohol. (See related technology in described in U.S. Pat. No. 10,537,659 which is incorporated herein by reference in its entirety. Twenty-four rats will undergo Roux-en-Y surgery. Four rats will have no sleeve placed and four will have a sleeve without GLP2. These are controls. Eight rats will have a low concentration GLP2 sleeve implanted into Roux limb and Eight rats will have a slightly higher concentration GLP2 sleeve implanted. X-rays will be taken at three intervals during the four weeks to monitor expansion. The animals will be sacrificed at 4 weeks for bowel length, histology, and markers. Again, postoperative diet toleration, weight gain, gut wall dilation and absence of intestinal obstruction will be evaluated. Segments of intestine with medicated IES will be compared to non-medicated IES for increases in length compared to segments containing non expanded IES.

Aim 3—Placement of sleeve in continuity (no Roux limb). Hypothesis Placing an expansion sleeve directly in continuity will demonstrate the safety of the device in vivo in a segment of intestine within the stream of chyme (no Roux-en-Y limb). A sleeve placed in continuity does not need a separate surgery to reconnect the Roux limb with the gastrointestinal tract. The benefit is obviously less surgery and less potential loss of intestinal length from surgical manipulation. Also, if the device can be used in intestinal continuity, then this is one step closer to the goal of placing such a device on a feeding tube to deploy it completely without surgery. We will use 22 rats to test sleeves in continuity. Four control rats will have a sham operation with permanent sutures place 20 mm apart on the outer intestinal wall with no sleeve placed. Nine rats will have sleeves placed in the small intestine and nine other rats will have sleeves with GLP2 placed in a segment of intestine. These sleeves will be secured with interrupted nonabsorbable suture. The animals will be sacrificed at 4 weeks for bowel length, histology, and markers. Segments from each group will be compared for increases in length.

### REFERENCES

1. DL, S. Short bowel syndrome in infants and children: an overview. Semin. Pediatr. Surg. 10, 49-55 (2001).
2. FR, D. et al. Enteral autonomy in pediatric short bowel syndrome: predictive factors one year after diagnosis. J. Pediatr. Surg. 50, 131-135 (2015).
3. A, B. Intestinal loop lengthening—a technique for increasing small intestinal length. J. Pediatr. Surg. 15, 145-151 (1980).
4. HB, K. et al. Serial transverse enteroplasty (STEP): a novel bowel lengthening procedure. J. Pediatr. Surg. 38, 425-429 (2003).
5. Greig, C. J., Oh, P. S., Gross, E. R. & Cowles, R. A. Retracing our STEPs: Four decades of progress in intestinal lengthening procedures for short bowel syndrome. Am. J. Surg. 217, 772-782 (2019).
6. RH, S. et al. Natural history of pediatric intestinal failure: initial report from the Pediatric Intestinal Failure Consortium. J. Pediatr. 161, (2012).
7. AU, S. et al. Enterogenesis in a clinically feasible model of mechanical small-bowel lengthening. Surgery 140, 212-220 (2006).
8. SD, S., AJ, F., KM, S., R, B. & MA, S. Longitudinal mechanical tension induces growth in the small bowel of juvenile rats. Gut 54, 1085-1090 (2005).
9. Huynh, N. et al. Spring-mediated distraction enterogenesis in-continuity. J. Pediatr. Surg. 51, (2016).
10. FR, D., PM, W., JJ, F., Y, F. & DH, T. A novel double-balloon catheter device for fully endoluminal intestinal lengthening. Pediatr. Surg. Int. 30, 1223-1229 (2014).
11. Portelli, K. I. et al. Intestinal adaptation following spring insertion into a roux limb in mice. J. Pediatr. Surg. 56, 346-351 (2021).
12. Clayton S, Alexander J S, Solitro G, White L, Villalba S, Winder E, Boudreaux M, Veerareddy P, Dong E, Minagar A, Dao H N, Sorrells D. Self-expanding intestinal expansion sleeves (IES) for short gut syndrome. Pediatr Surg Int. 2022 January; 38 (1): 75-81. doi: 10.1007/s00383-021-05024-8. Epub 2021 Oct. 28. PMID: 34709433.
13. Dubrovsky, G., Huynh, N., Thomas, A. L., Shekherdimian, S. & Dunn, J. C. Intestinal lengthening via multiple in-continuity springs. J. Pediatr. Surg. 54, (2019).
14. Mouillot T, Boehm V, Treton X, Ferrandi E, Kapel N, Cazals-Hatem D, Joly F. Small-Bowel Adaptation: A Case of Morphological Changes Induced by Teduglutide in Short-Bowel Syndrome With Intestinal Failure. JPEN J Parenter Enteral Nutr. 2020 July; 44 (5): 940-943. doi: 10.1002/jpen.1805. Epub 2020 Mar. 18. PMID: 32187383.

### Example 2

This disclosure describes an apparatus and a method for deployment of a woven nylon, plastic or metal mesh tube which has the property that when expanded diametrically the length of the tube is reduced proportionately, under an embodiment. This allows the tube to be placed onto a deployment sheath which holds the tube in the ‘open’ conformation which is 30% shorter when in this conformation. The tube is then inserted into a region of gut and the ends are sutured into the gut wall. The stent ends are then compressed together slightly which allows the coil to release from the stent and the sheath is removed. When the stent is ‘relaxed’ it attempts to lengthen against the gut wall and produces significant longitudinal stretch which immediately lengthens the gut by ˜20%. This is not the full lengthening capacity however and if allowed to fully expand, the gut segment expanded by the gut would be lengthened by up to 50%. After gut remodeling, this would achieve an improvement of gut length which is the significant problem in short bowel syndrome.

FIGS. 4A-4I describe the introduction of a pre shortened segment of the mesh tube (Figure B), which decreases the tube length, but increases its diameter. In this shortened but widened format the tube is introduced into a segment of gut (Figure C). Following its deployment into the segment of the gut which is to be extended, the shortened tube on its guide is then sutured into place (Figure D and E) in the gut while the gut is under normal tension. Following suturing at both ends (Figure F), the stent is 7 cm in length (Figure G). After the deployment guide is removed (Figure H) the tube increases in length immediately to 20-25% longer than the initial loading length (Figure I, 9 cm). This produces significant lengthening tension in the gut. This is intended to lead to a persistent lengthening by longitudinal tension in the gut which over time is intended to produce an extension of the gut. It The gut fully remodels during the placement of this device to achieve clinical benefit.

### Example 3

PURPOSE. Many disease processes (necrotizing enterocolitis, caustic esophageal injury, malrotation with volvulus), can result in short-gut syndrome (SGS) where remnant intestinal segments may dilate axially, but rarely elongate longitudinally. Here we mechanically characterize a novel model of a self-expanding mesh prototype intestinal expanding sleeve (IES) for use in SGS.

METHODS. Gut lengthening was achieved using a proprietary cylindrical layered polyethylene terephthalate IES device with helicoid trusses with isometric ends. The IES is pre-contracted by diametric expansion, deployed into the gut and anchored with bioabsorbable sutures. IES expansion to its equilibrium dimension maintained longitudinal gut tension, which may permit remodeling, increased absorptive surface area while preserving vascular and nervous supplies. We performed mechanical testing to obtain the effective force-displacement characterization achieved on these prototypes and evaluated minimal numbers of sutures needed for its anchoring. Furthermore, we deployed these devices in small and large intestines of New Zealand White rabbits, measured IES length-tension relationships and measured post-implant gut expansion ex vivo. Histology of the gut before and after implantation was also evaluated.

RESULTS. Longitudinal tension using IES did not result in suture failure. Maximum IES suture mechanical loading was tested using 4-6 sutures; we found similar failure loads of 2.95±0.64, 4±1.9 and 3.16±0.24 Newtons for 4, 6 and 8 sutures respectively (n=3, n.s). Pre-contracted IES tubes were deployed at 67±4% of initial length (i.l.); in the large bowel these expanded significantly to 81.5±3.7% of i.l. (p=0.014, n=4). In the small bowel, pre-contracted IES were 61±3.8% of i.l.; these expanded significantly to 82.7±7.4% of i.l. (p=0.0009, n=6). This resulted in an immediate 24±7.8% and 36.2±11% increase in gut length when deployed in large and small bowels respectively with maintained longitudinal tension. Maintained IES induced tension produced gut wall thinning; gut histopathological evaluation is currently under evaluation.

CONCLUSION. IES is a versatile platform for gaining length in SGS, which may be simply deployed via feeding tubes. Our results need further validation for biocompatibility and mechanical characterization to optimize use in gut expansion.

### BACKGROUND

Pediatric surgeons often encounter patients with intestinal failure due to inadequate intestinal length. These patients are challenging because the adaptive process for reaching enteral goals is slow. Patients with short gut compensate with gastroparesis and slow dilation of the intestinal diameter.¹ This process may take weeks, months or years requiring supplemental parenteral nutrition for adequate growth.² Native elongation of the short intestine usually is frequently limited. Two surgical elongation procedures have been developed—the Bianchi³ and serial transverse enteroplasty (STEP) procedure⁴. Both procedures require that the patient has developed sufficient dilation of bowel diameter.⁵ The medical care of these patients is encumbered by daily needs for total parenteral nutrition (TPN), central line access, and metabolic derangements [6].

One possible solution to managing this clinical dilemma is to initiate an intestinal elongation strategy early. Distraction enterogenesis (DE) with various devices has been shown in animal models to enable elongation of the intestine [7]. These devices cause longitudinal tension on a segment of intestine resulting in elongation of the segment by 50% or more [8]. Some of these devices include springs [9] and balloon devices [10]. Initial studies have these devices placed out of continuity of the gastrointestinal tract such as in a Roux limb [11]. The spring device has so far been successfully placed in continuity in a rat model [12]. The potential improvement in the clinical course of these patients is tremendous with decreased days on TPN, decreased need for central access and decreased line infections.

There are several potential benefits/advantages of the expanding intestinal sleeves over other prototypes. The device is porous making successful placement in continuity likely. The contracted device expands radially in proportion to the amount of shortening in length of the IES. The radial expansion should make the device easier to secure to the intestine in a future noninvasive model. Lastly, the fully expanded sleeve has a decreased diameter making eventual passage of the IES in the stool more likely.

Our DE device is an implantable porous mesh sleeve intended to be attached at each end to the intestine with absorbable sutures. The device is designed to exert a linear force curve as it expands lengthening the intestine. The segment would eventually be passed in stool once the absorbable sutures have dissolved. Ultimately, to save the patient operations and potential intestinal length, the device would be fixed to the end of a long transpyloric feeding tube and deployed without surgery. Multiple deployments of these sleeves should be possible. In this study, we sought to evaluate the achieved force-strain relationship in the built prototypes, evaluate the achievable tensioning force in relation to the number of sutures used for its anchoring, and present an initial feasibility of placing the novel sleeve in rabbit intestine. We hypothesize that an expansion greater than 40% of the initial device length is achievable with no more than six sutures.

A nonoperative deployment of Intestinal Expansion Sleeve is used under an embodiment. The patient with short bowel syndrome has an MRI demonstrating the current state of small intestinal size or diameter. The intestinal expansion sleeve is custom made to be about 30% larger when contracted than the MRI measurement of intestine. This sleeve is placed uncontracted on the end of a long feeding tube (i.e. duotube). The duotube is placed per usual standard fashion through the patient's nose and down into stomach. Over several days the end to the tube migrates past the duodenum into the proximal small bowel. This can be verified with contrast injection under fluoroscopy. When the end of the feeding tube has migrated to the ‘target zone’ area of small bowel, the device at the end of the feeding tube is deployed. Deployment constitutes contraction of the sleeve to the size which will facilitate attachment of the device to the intestinal wall. The expanded device separates from the feeding tube and the device will then exert linear force on the intestinal wall causing distraction enterogenesis. Deployment of multiple sleeves per application or several applications are needed to acquire significant functional lengthening of the intestine. However, these simultaneous or staggered deployments of expansion sleeves should result in increases in functional intestinal mass.

### Methods

### Sleeve Preparation and Characterization:

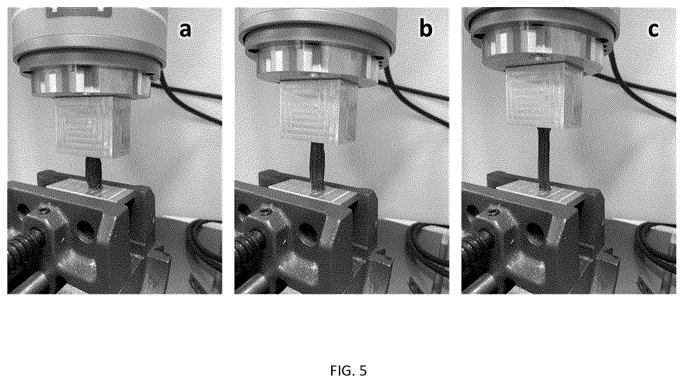

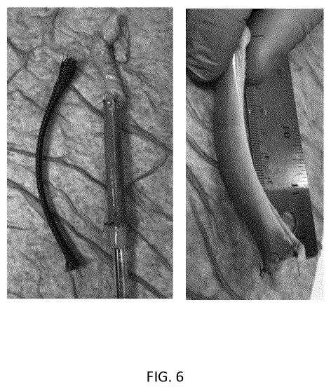

Intestinal expansion sleeves (IES) were produced as cylindrical layered polyethylene tubes with helical trusses. The ends of the sleeves were heat-treated to smooth the edges. Edges of sleeves were inspected and milled smooth as needed. The sleeve lengths were measured individually in their native form (initial length, ‘i.l.’). Mechanical characterization was performed using an Instron 8874 Biaxial Servo Hydraulic Fatigue Testing System, applying a cycle of compression followed by expansion at a rate of 50 mm/min (see FIG. 5). With reference to the nominal length of the DE devices, force values were recorded during the expansion phase at strain values 10, 20, 30, 40 and 50%.

FIG. 5 shows a mechanical characterization of the DE device during the expansion phase at strains of 50% (FIG. 5A), 25% (FIG. 5B) and at its nominal length (FIG. 5C).

### Implantation of the Sleeves:

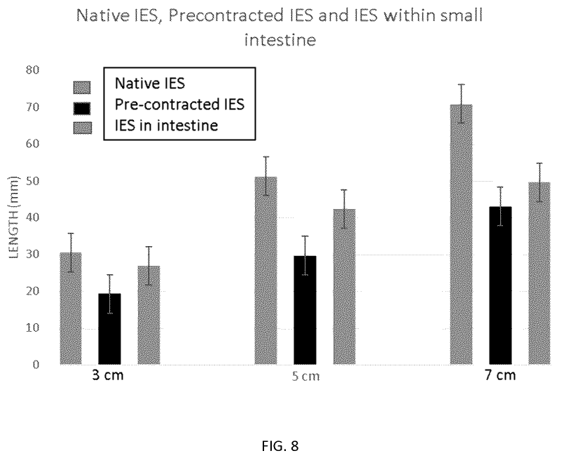

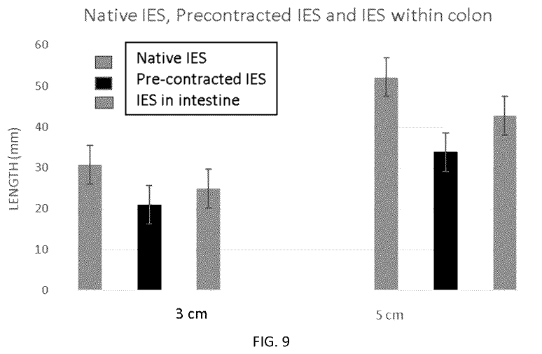

Using small and large intestines from New Zealand White rabbits, several test sleeves were implanted. Intestines were harvested from recently sacrificed rabbits and lumen washed with isotonic saline. Measurements of the intestine in native form were obtained, and sleeves then placed over a 0.8 cm plastic tube with an end tapered to 3 mm (FIG. 6). The placement of the sleeve over the tubing resulted in shortening of the sleeve to its pre-contracted form which was recorded. An enterotomy was made in intestinal segment and the tube containing the pre-contracted sleeve was placed into the intestinal segment. Four sutures (4-0 polydioxanone, Ethicon) were placed at both ends of the IES to secure the sleeve inside the intestinal wall in the pre-contracted state. Removal of the pipette resulted in immediate partial expansion of the sleeve within the intestinal segment; the extent of bowel length extension was measured using calipers. These data are shown in FIGS. 8 and 9.

FIG. 6 shows a representative sleeve and introduction into intestine (left). Intestine after expansion of the sleeve (right).

### Mechanical Testing of the Bowel/Device Connection:

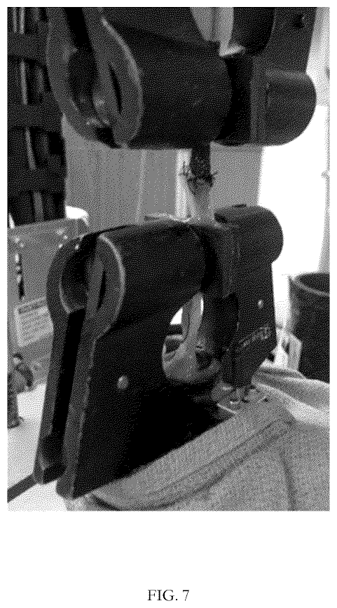

Intestinal segments instrumented with the DE device were tested to determine maximum force prior to failure in relation to the number of sutures used for the anchoring of the DE device. The testing was performed on the same Instron machine used for the characterization. The specimens, consisting of a DE device (IES) connected to a bowel segment were held in place by two pneumatic grips at 25 psi of pressure as shown in FIG. 7. Sleeve placement in the rabbit small intestine versus length of IES (n=3 for each group). Testing to failure was performed by delivering tension to the specimens at a rate of 50 mm/min until failure. The three configurations tested, included DE devices connected with 4, 6 or 8 sutures. Load displacement data were recorded at a frequency of 100 Hz and for every 0.1N increments. The peak load measured during testing was considered as the failure load for the specimen.

FIG. 7 shows mechanical testing of IES bowel connection, IES device at top (blue) is sutured to intestinal wall (lower section) and load failure performance analyzed.

### Results

### Mechanical Characterization of the Created Prototypes

The tested prototypes exerted forces that varied from 0.73±0.28N for the 10% strain to a peak of 6.44±1.05N for the 50% strain (p=0.01, see Table 1). The 30 and 40% strains, respectively with loads of 2.31±0.35N and 2.84±0.36N were significantly different (p=0.01) but comparable in amplitude to the failure loads later measured for the bowel.

TABLE 1
Load values [N] recorded at various strain values for
each tested nominal length of the DE device.

| DE nominal | | | | | |
|---|---|---|---|---|---|
| length | Strain | | | | |
| [mm] | 10% | 20% | 30% | 40% | 50% |
| 30 | 0.46 | 1.61 | 2.32 | 2.82 | 6.08 |
| 30 | 0.62 | 2.07 | 2.84 | 3.31 | 7.29 |
| 30 | 0.85 | 2.37 | 2.99 | 3.54 | 7.59 |
| 40 | 0.10 | 1.10 | 1.82 | 2.26 | 3.99 |
| 40 | 0.70 | 1.76 | 2.14 | 2.83 | 7.63 |
| 40 | 1.01 | 1.83 | 2.18 | 2.78 | 6.57 |
| 50 | 0.98 | 1.91 | 2.23 | 2.78 | 6.41 |
| 50 | 0.94 | 1.77 | 2.15 | 2.55 | 5.78 |
| 50 | 0.91 | 1.80 | 2.10 | 2.67 | 6.64 |
| Average | 0.73 | 1.80 | 2.31 | 2.84 | 6.44 |
| SD | 0.28 | 0.32 | 0.35 | 0.36 | 1.06 |

The bowel alone has shown a failure load of 2.48±0.17N that was not different from the failure load of 2.95±0.52N found for the IES/bowel connection performed with 4 sutures (p=0.10). Compared to the connection with 4 sutures, the increment to 8 resulted in a failure load of 3.16±0.20N that was found not significant different (p=0.31, see Table 2). The failure of the system under tension revealed that failure was mainly localized in the bowels and was independent of suture numbers.

TABLE 2
Bowel/device connection

| number of | Failure | Localization of the |
|---|---|---|
| sutures | load [N] | failure formation |
| intact bowel | 2.75 | Proximity of the grip |
| intact bowel | 2.31 | Mid-bowel |
| intact bowel | 2.38 | Mid-bowel |
| Intact bowel | 2.49 | Mid-bowel |
| 4 | 2.85 | bowel |
| 4 | 3.64 | bowel |
| 4 | 2.37 | stitches |
| 6 | 2.46 | bowel |
| 6 | 3.33 | bowel |
| 6 | 6.25 | stitches |
| 8 | 3.42 | stitches |
| 8 | 2.94 | bowel |
| 8 | 3.12 | bowel |

### Animal Model

Six (6) sleeves were placed in colons and 9 sleeves were placed in small bowels. Measurements were taken by a caliper and lengths recorded in mm. The sleeves used in the colon showed a pre-deployment decrease in length of 67±3.6% of i.l. The sleeves expanded in the colon to 81.6±3% of i.l. of the sleeve (p=0.014, n=6). In the small bowel pre-contracted IES were 60.6±3.7% of i.l.; these expanded significantly to 80.2±8.4% of i.l. (p=0.0009, n=9). This resulted in an immediate 24±7.8% and 36.2±11% increase in gut length of the colon and small bowel respectively.

FIG. 8 shows sleeve placement data in the rabbit small intestine versus length of IES (n=3 for each group).

FIG. 9 shows sleeve placement data in the rabbit colon versus length of IES (n=three for each group).

Average increase of intestinal length for 3 cm and 5 cm sleeves in the small intestine was 39±3% and 44±13% respectively. Interestingly, the percent increase in length for 7 cm was only 16±8%. The colon data were similar (FIG. 9, n=3 in each group). The IES colon percent increase in i.l. was 19±8.5% for 3 cm sleeve and 26.7±3.5% for 5 cm.

As already seen above in FIGS. 2A-2D, histology of the small intestine (left) with IES showed significant wall thinning in all layers of the small and large intestines in response to dilation of the lumens with IES. Figure A) Small intestine undilated and Figure B) dilated. Figure C) Large intestine undilated and Figure D) dilated. Bar=300 μm. Comparisons made by t-test between non-expanded and expanded states for total thickness and each tissue layer.

We accomplished load testing on IES sutures in small bowel with 4, 6 or 8 sutures. Failure under load as defined by a sudden drop in tension was seen at 2.95±0.64, 4±1.9 and 3.16±0.24 Newtons for each respective number of sutures. There was no significant difference between these groups regarding failure load. There was no apparent relationship between the number of sutures and the failure load indicating that using four sutures to secure the IES device in the intestine may be safe and may not be improved by additional suturing. The failure load of the small intestine alone was 4±1.5 Newtons.

The expansion force was measured for sleeve lengths of 3, 5, and 7 cm for the small intestine. We found that the expansion force at 10% differed significantly from 20%, 30%, 40% and 50% at a length of 30 mm. Similarly, at 50 mm, the expansion force again differed significantly between 10% and other values 20%, 30%, 40% and 50% values of compression. At the intermediate length of 40 mm, 10% compression was only significantly different from the 50% compression.

### Discussion

Short bowel syndrome (SBS) is considered the most common cause of intestinal failure in pediatric patients. Loss of small bowel length results in inadequate nutrient absorption due to a lack of functional surface area, leading to increased morbidity and mortality [6]. The most common etiologies of short bowel syndrome in infant and pediatric patients include necrotizing enterocolitis, abdominal wall defects, jejunal ileal atresia, and mid gut volvulus [1]. Bowel lengthening surgical procedures have been developed, however they carry substantial risks for morbidity due to their invasive nature and exclude patients with undesirable anatomy [13-15]. Other methods of treatment include medications to slow intestinal transit and parenteral nutrition. Most patients are dependent on the parenteral nutrition route, which leads to risks such as sepsis, metabolic derangements and hepatic dysfunction. Remaining bowel length has been shown to positively correlate with the ability to wean from parenteral nutrition [2, 16, 17]. Distraction enterogenesis (DE) is a recently developed method whereby small intestine elongation is accomplished by the use of longitudinal mechanical force, which has been hypothesized to provide a therapeutic option for those with SBS [7, 8, 18]. The main challenge with DE has been developing a method which minimizes axial expansion while maximizing longitudinal lengthening. Previous successful methods of attaining longitudinal bowel lengthening include hydraulic pistons, spring loaded devices, and osmotic distension. Unfortunately, all of these methods have been limited by their transmission of axial force against the bowel wall, which leads to risks such as perforation and necrosis secondary to compression of the blood supply of the bowel wall [10]. The excessive axial force component of these previous methods has led to significant challenges in translating these basic science experiments to clinically applicable technology.

Despite its challenges, DE has shown promise in obtaining sustained intestinal lengthening with concomitant functionality. Previous studies have characterized this preserved functionality by the presence of mesenteric neovascularization, muscular hypertrophy, increased epithelial cell proliferation, and increased villus height and crypt depth.^{19,20} Early studies testing DE in pigs required creation of blind-ending segments of intestine^{8,18} or the use of vessels loops or full thickness sutures to facilitate mechanical force transmission to the bowel wall.¹⁹ These methods required multiple separate open operations to both place and remove the devices or restore intestinal continuity, which adds substantial surgical complexity and potentially results in the loss of gained intestinal length after anastomosis creation.²¹ Further advancements led to the creation of devices which could provide longitudinal force via reversible endoluminal attachments. This improved delivery system obviated the need for additional procedures to remove the device. Challenges with this new approach included an efficient delivery system and atraumatic distraction attachments.¹⁰

Mechanical data (Table 2) show that average failure load for native rabbit small intestine was 2.48±0.19. Failure load with four fixation sutures was similar and ⅔ of failures occurred in bowel and not the connection of the tensiometer to intestine (n=9). Increasing the number of sutures to the IES device did not significantly affect the failure load. These data support four fixation sutures for the IES device.

Our strain data on prototypes of varying lengths show that the sleeves exceed the failure load of rabbit intestine when compressed more than 30% (Table 1). This will require some widening of helical trusses and dampening of the load values of IES prior to placing in vivo in New Zealand white rabbits.

Our data demonstrate a significant immediate expansion of the intestine (small bowel or colon) and the resilience of the suture fixation of the IES. Mechanical testing also demonstrated the load properties of the intestine before elongation by the IES. This information is vital to the design of IES devices. Optimally, the force (N) of expansion of the IES should be less than the failure load of the intestine. Histology shows that stretching of the intestinal wall reduces wall thickness and flattening of individual wall layers, which may influence perfusion and wall tension; these factors need to be more fully evaluated in additional studies. Designing IES devices with sub intestine failure load force dynamics will decrease the potential for intestinal perforation.

When we compare different lengths of IES, the initial expansion benefit appears to dimmish at the 7 cm (16±8%) vs 5 cm (44±13%) length. This seems to indicate an optimal length for the IES and longer lengths are not always better. Key to gaining maximum length may be deployment of multiple IES devices or serial deployments over time. Future studies will focus on optimal length of IES and potential for multiple deployments.

Distraction enterogenesis has the potential to treat short bowel syndrome. Our IES offers multiple improvements compared to prior devices. The sleeve is porous and should be deployable in continuity with GI tract. Also, the IES will not require removal as it will be held in place with absorbable sutures. The device has limited axial expansion and excellent linear expansion as shown by this study. Lastly, the future use of IES may enable device to be deployed over a feeding catheter as well as coated with drugs to promote healing or decrease inflammation.

### REFERENCES

1. DL, S. Short bowel syndrome in infants and children: an overview. Semin. Pediatr. Surg. 10, 49-55 (2001).
2. FR, D. et al. Enteral autonomy in pediatric short bowel syndrome: predictive factors one year after diagnosis. J. Pediatr. Surg. 50, 131-135 (2015).
3. A, B. Intestinal loop lengthening—a technique for increasing small intestinal length. J. Pediatr. Surg. 15, 145-151 (1980).
4. HB, K. et al. Serial transverse enteroplasty (STEP): a novel bowel lengthening procedure. J. Pediatr. Surg. 38, 425-429 (2003).
5. Greig, C. J., Oh, P. S., Gross, E. R. & Cowles, R. A. Retracing our STEPs: Four decades of progress in intestinal lengthening procedures for short bowel syndrome. Am. J. Surg. 217, 772-782 (2019).
6. RH, S. et al. Natural history of pediatric intestinal failure: initial report from the Pediatric Intestinal Failure Consortium. J. Pediatr. 161, (2012).
7. AU, S. et al. Enterogenesis in a clinically feasible model of mechanical small-bowel lengthening. Surgery 140, 212-220 (2006).
8. SD, S., AJ, F., KM, S., R, B. & MA, S. Longitudinal mechanical tension induces growth in the small bowel of juvenile rats. Gut 54, 1085-1090 (2005).
9. Huynh, N. et al. Spring-mediated distraction enterogenesis in-continuity. J. Pediatr. Surg. 51, (2016).
10. FR, D., PM, W., JJ, F., Y, F. & DH, T. A novel double-balloon catheter device for fully endoluminal intestinal lengthening. Pediatr. Surg. Int. 30, 1223-1229 (2014).
11. Portelli, K. I. et al. Intestinal adaptation following spring insertion into a roux limb in mice. J. Pediatr. Surg. 56, 346-351 (2021).
12. Dubrovsky, G., Huynh, N., Thomas, A. L., Shekherdimian, S. & Dunn, J. C. Intestinal lengthening via multiple in-continuity springs. J. Pediatr. Surg. 54, (2019).
13. EA, M., PI, B. & DH, T. Redilation of bowel after intestinal lengthening procedures—an indicator for poor outcome. J. Pediatr. Surg. 46, 145-149 (2011).
14. TE, G., HB, C., JF, V. & MS, D. Staple line ulcers: a cause of chronic GI bleeding following STEP procedure. J. Pediatr. Surg. 48, (2013).
15. D, S. et al. Comparison of intestinal lengthening procedures for patients with short bowel syndrome. Ann. Surg. 246, 593-601 (2007).
16. EM, F. et al. Neonates with short bowel syndrome: an optimistic future for parenteral nutrition independence. JAMA Surg. 149, 663-670 (2014).
17. AU, S. et al. Pediatric short bowel syndrome: redefining predictors of success. Ann. Surg. 242, 403-412 (2005).
18. J, P., DP, P., BM, W., JB, A. & JC, D. Enterogenesis by mechanical lengthening: morphology and function of the lengthened small intestine. J. Pediatr. Surg. 39, 1823-1827 (2004).
19. MW, R. et al. Mesenteric neovascularization with distraction-induced intestinal growth: enterogenesis. Pediatr. Surg. Int. 29, 33-39 (2013).
20. M, O., HM, M. & DH, T. Distraction induced enterogenesis: a unique mouse model using polyethylene glycol. J. Surg. Res. 170, 41-47 (2011).
21. R, S., T, Z., S, B. & JC, D. Restoration of mechanically lengthened jejunum into intestinal continuity in rats. J. Pediatr. Surg. 46, 2321-2326 (2011).

### Example 4

Short bowel syndrome (SBS) is characterized by insufficient intestinal length leading to malabsorption and malnutrition, requiring supplemental parenteral nutrition. In a year, the cost of parenteral nutrition in the US market can be $2.5 billion. 80% of this cost was from neonates with a significant portion due to short bowel syndrome. Therefore, functional intestinal elongation to expedite the process of bowel adaptation could save enormous costs associated with the care of this population. The normal process of bowel adaptation occurs through intestinal dilation, instead of elongation, and slowed gastric emptying, but patients still often require long term parenteral nutrition.

Surgical options to lengthen the bowel pose significant risks and often provide limited expansion. The current options generally provide about 20 cm of length and more than 50% of patients continue to require parenteral nutrition. Distraction enterogenesis is a proposed technique to induce in vivo intestinal lengthening for SBS. An evaluation of the efficiency of the intestinal expansion sleeve (IES) in lengthening the bowel is disclosed herein.

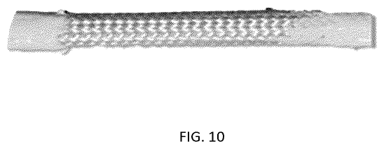

Springs have successfully been placed in continuity in pig and rat intestine with demonstrated elongation but had complications such as perforation. A new device (or sleeve as seen in FIG. 10) is disclosed herein to initiate endoluminal elongation of the intestine early by creating longitudinal tension on the bowel. The sleeve is a woven cylindrical device with isometric ends being used to achieve mechanical lengthening via DE. Under an embodiment, the sleeve is implantable with absorbable sutures. As it elongates, the sleeve diameter decreases.

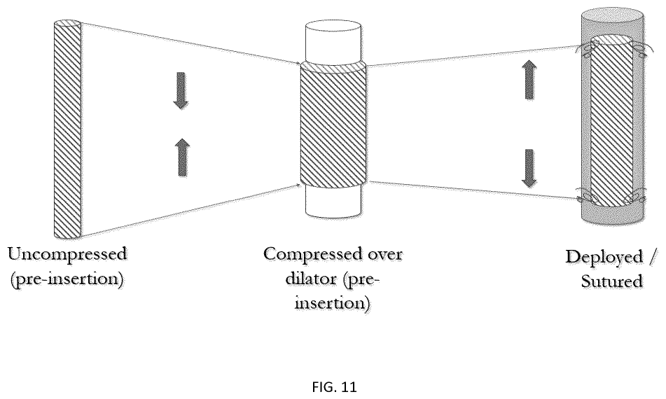

FIG. 11 illustrates an embodiment of the expansion sleeve. FIG. 11 shows the sleeve (i) in an uncompressed state, (ii) in compressed state over dilator (pre-insertion), and (iii) in a deployed state sutured to an interior of a bowel segment.

### Implantation:

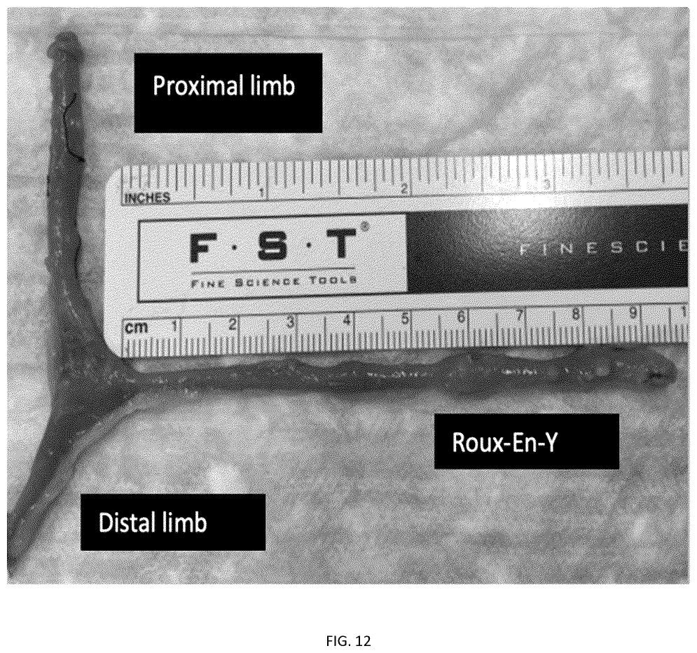

A Roux-en-Y (see FIG. 12) in the jejunum of 6 Sprague Dawley rats was created for isolated IES deployment.
The contracted device was inserted into the Roux limb out of chyme or enteral flow and then fixated with silk sutures to the bowel.
Anastomoses were made to reconnect the bowel and the abdomen was closed

### Deployment:

Device longitudinally expands after support and suture are absorbed. In this case, the rigid support was a piece of Bucatini noodle which softens and passes once in vivo
We allowed expansion for one month.
Thereafter, the rats were sacrificed, and we removed Roux-En-Y segments for histological evaluation.

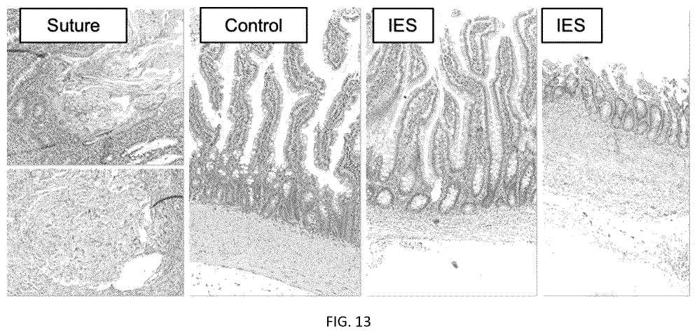

FIG. 13 is a representative sample of a control and 2 of the IES histology results

One can see post op changes consistent with sutures localized on both control and IES roux limbs. (Of note, the adjacent bowel mucosa is preserved). The control shows unremarkable intact small bowel. The IES samples showed variation in mucosal and submucosal integrity and varying bowel wall thickness in response to IES lengthening. This first IES showed that despite being stretched the mucosal villi maintained length and integrity. The far right IES shows partial mucosal erosion. Importantly, the basal regenerative layer of the mucosa is preserved. Therefore, we would expect regeneration of the mucosa after the lengthening device has passed. Overall, all bowel functional layers and attributes were maintained through distraction enterogenesis in the study.

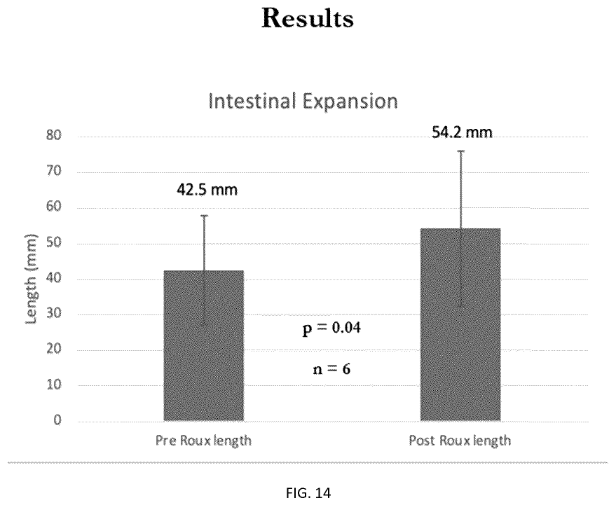

As shown in FIG. 14, a paired t-test was performed to compare initial and final roux limb lengths. Deployment of IES devices over one month produced statistically significant lengthening of 42.5±15 mm to 54.2±22 mm (p=. 04, n=6). This represents a nearly 30% elongation compared to the initial length.

### REFERENCES

1. Clayton S D et al (2021) Self-expanding intestinal expansion sleeves (IES) for short gut syndrome. Pediatr Surg Int. 38 (1), 75-81
2. Meyer, H., Clayton, S. Distraction vaginogenesis: Preliminary results using a novel method for vaginal canal explanation in rats. Bioengineering. 2023 Mar. 12; 10 (3): 351. PMID 36978742
3. Sigalet D L (2001) Short bowel syndrome in infants and children: an overview. Semin Pediatr Surg 10:49-55
4. Demehri F R et al (2015) Enteral autonomy in pediatric short bowel syndrome: predictive factors one year after diagnosis. J Pediatr Surg 50:131-135
5. Bianchi A (1980) Intestinal loop lengthening—a technique for increasing small intestinal length. J Pediatr Surg 15:145-151
6. Kim H B et al (2003) Serial transverse enteroplasty (STEP): a novel bowel lengthening procedure. J Pediatr Surg 38:425-429
7. Greig C J et al (2019) Retracing our STEPs: Four decades of progress in intestinal lengthening procedures for short bowel syndrome. Am J Surg 217:772-782
8. Squires R H et al (2012) Natural history of pediatric intestinal fail-ure: initial report from the Pediatric Intestinal Failure Consortium. J Pediatr. https://doi.org/10.1016/j.jpeds.2012.03.062
9. Spencer A U et al (2006) Enterogenesis in a clinically feasi-ble model of mechanical small-bowel lengthening. Surgery 140:212-220
10. Safford S D et al (2005) Longitudinal mechanical tension induces growth in the small bowel of juvenile rats. Gut 54:1085-1090
11. Huynh N et al (2016) Spring-mediated distraction enterogenesis in-continuity. J Pediatr Surg. https://doi.org/10.1016/j.jpedsurg.2016.09.024

### Example 5

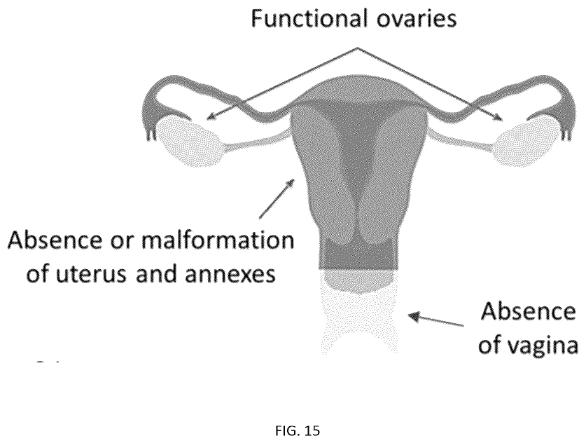

Mayer-Rokitansky-Küster-Hauser (MRKH) Syndrome results in females born without uterus and/or vaginal canal. (See FIG. 15). This syndrome can have severe impacts on sexual function, self-esteem, and quality of life.

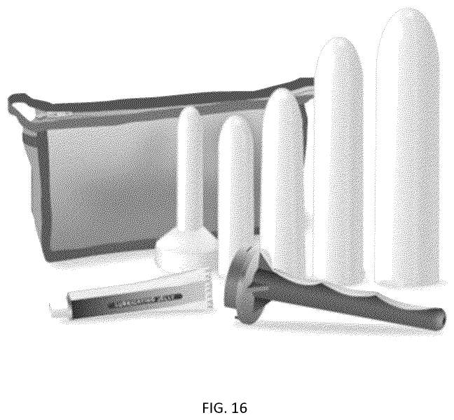

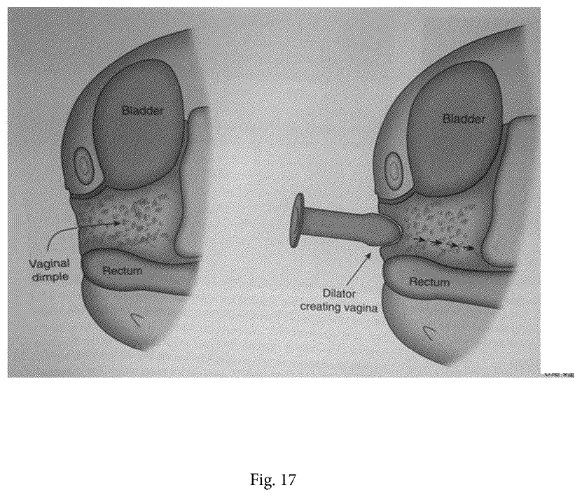

Mechanical Serial Dilation is one treatment option. It is a first-line, low cost, generally safe treatment. Timeline of treatment depends on initial vaginal canal length but may comprise 3-6 months. A drawback of the treatment is that it requires a heavily motivated patient. (See FIGS. 16-17).

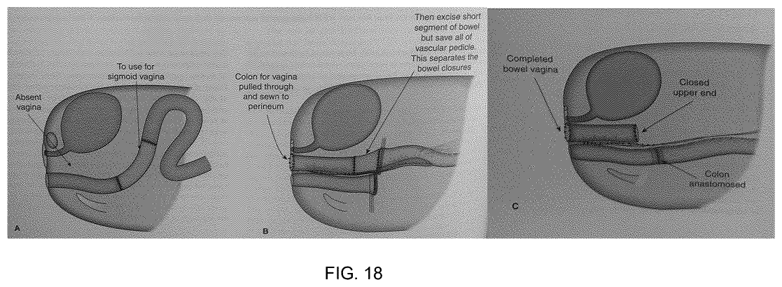

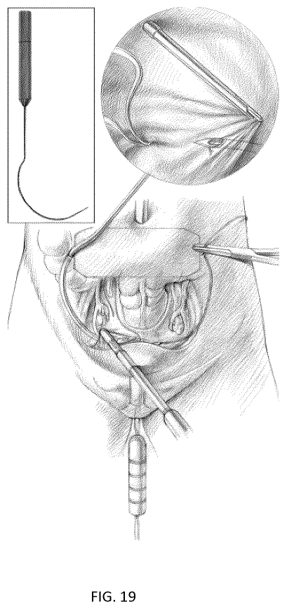

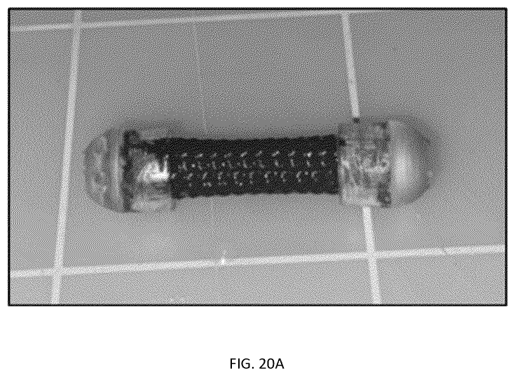

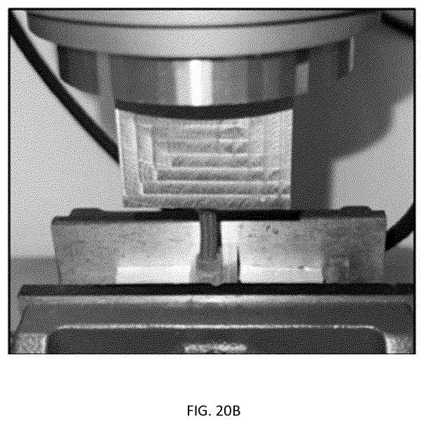

Surgical Vaginoplasty is another treatment option. Such procedures include the Vecchietti procedure, the Abbe-McIndoe procedure, and the Intestinal vaginoplasty. However such treatments involve high complication rates, and many patients need secondary surgeries. (See FIGS. 18-19).

Vaginal atresia is seen in genetic disorders such as Mayer-Rokitansky-Küster-Hauser (MRKH) syndrome, which can cause significant sexual dysfunction. Current treatments include surgical reconstruction or mechanical dilation of the vaginal canal. Mechanical dilation requires patients to be highly motivated and compliant while surgical reconstruction has high rates of complications. This study evaluated a novel vaginal expansion sleeve (VES) method as an alternative treatment for vaginal atresia. The proprietary cylindrical VES is a spring-like device consisting of polyethylene terephthalate helicoid trusses capped at each end with a fixed diameter resin cap for fixation within tissues. Following the development of the VES and mechanical characterization of the force-length relationships within the device, we deployed the VES in Sprague Dawley rat vaginas anchored with nonabsorbable sutures. We measured the VES length-tension relationships and post-implant vaginal canal expansion ex vivo. Vaginal histology was examined before and after implantation of the VES devices. Testing of 30 mm sleeves without caps resulted in an expansion force of 11.7±3.4 N and 2.0±0.1 N at 50% and 40%, respectively. The implanted 20 mm VES resulted in 5.36 mm±1.18 expansion of the vaginal canal, a 32.5±23.6% increase (p=0.004, Student t test). Histological evaluation of the VES implanted tissue showed a significant thinning of the vaginal wall when the VES was implanted. The novel VES device resulted in a significant expansion of the vaginal canal ex vivo. The VES device represents a unique alternative to traditional mechanical dilation therapy in the treatment of vaginal atresia and represents a useful platform for the mechanical distension of hollow compartments, which avoids reconstructive surgeries and progressive dilator approaches.

### INTRODUCTION

Vaginal atresia is a congenital absence or underdevelopment of the vaginal canal and is commonly seen in genetic disorders such as Mayer-Rokitansky-Küster-Hauser (MRKH) syndrome or complete androgen insensitivity syndrome (CAIS) [1,2]. MRKH syndrome is a rare congenital disorder that occurs in approximately one in 4000-5000 female births. Females with MRKH have vaginal aplasia and other Mullerian (i.e., paramesonephric) duct abnormalities. Women with MRKH typically lack a fully uterus and vaginal canal, but are otherwise externally phenotypically and genotypically normal with all other secondary sex characteristics [3,4]. CAIS is another congenital malformation that occurs in about one in every 20,000 male births. CAIS is characterized by undescended testes and phenotypically female external genitalia in genotypic males. In CAIS, the vaginal canal is shorter (less than 8 cm) than that in normal women and is blind ending [5,6]. Partial androgen insensitivity syndrome (PAIS) is also characterized by atypical genitalia, which may also require reconstruction based on the gender identity of the patient. The absence of a vaginal canal in MRKH, CAIS, and PAIS can result in severe sexual dysfunction in adolescence and adulthood with profound psychosocial stresses [3]. Currently, available treatments for such forms of vaginal atresia include reconstructive surgical and non-surgical options.

The current first-line treatment for vaginal agenesis is serial mechanical dilation of the vaginal canal where pressure and hard dilators are inserted incrementally into the vagina to progressively increase both the length and diameter of the vaginal canal [7]. Frank first described dilation therapy in 1938, which has since become more widely used [8]. Procedural success, determined as a functional vaginal canal, does not depend on the starting vaginal length, but depends on significant patient compliance and support to perform 30-min of dilation up to three times a day [2]. Treatment can take an average of about six months when compliant, with one study of 245 patients describing a range of two to nineteen months [4]. Because of this need for patient cooperation, previous literature has suggested that therapy begins after the patient is evaluated by a psychologist for emotional maturity and is determined to have adequate intrinsic motivation to avoid a psychological barrier to dilator treatment or to avoid exacerbation of pre-existing psychological hardships as a result of their diagnosis [4,9]. Primary dilation is considered first-line due to lower morbidity and costs than surgical therapy [2,7,10,11]. Common complications are less severe than those of surgery and consist of urethral irritation and urinary leakage [2,4]. Despite being safer and more cost-effective, dilation is not universally successful, and some cases require a surgical approach.

Various techniques for surgical vaginoplasty exist. The type of procedure a patient undergoes depends on several factors including surgeon preference, the patient's clinical condition, and the patient's age [12]. A previous study of 131 patients found that surgical vaginoplasty had a 40% higher rate of complications compared to primary dilation therapy [13]. Many of these complications will subsequently require secondary operations for correction [2]. One laparoscopic method is the Vecchietti procedure, which utilizes a small olive-shaped bead attached to threads connected to a traction device on the abdominal wall that are then tightened about 1 cm per day for 7-10 days or until a satisfactory length is reached (7-10 cm) [1,14,15]. Due to the pain associated with continuous traction, the patient must stay in the hospital throughout the traction process [15]. Moreover, potential significant complications such as bladder and rectal lesions are possible due to the limited retrovesicorectal space where the threads are placed [14,15].

The Abbe-McIndoe procedure, an open method, consists of dissecting out the potential space of the neovagina followed by a skin graft to create the new vaginal wall. This procedure may be accompanied by serious complications such as infection from the contact between the vagina and peritoneal cavity, infection of the graft, and neovaginal stenosis [14-16]. A more complicated and invasive method is intestinal vaginoplasty, which uses a segment of the bowel, usually the sigmoid, ileum, or jejunum, to create a neo-vagina [14]. Serious complications of the intestinal vaginoplasty include intra-abdominal hemorrhage, intestinal obstructions, and prolapse of the vaginoplasty architecture [14-16]. One major advantage that sets the intestinal vaginoplasty apart from the Vecchietti and Abbe-McIndoe procedures is the lack of possible vaginal contraction post-operatively. Therefore, the patient is not required to complete manual dilation after surgery to main-tain vaginal patency [14,15]. Generally, other forms of vaginoplasty require mechanical dilation therapy after surgery to maintain vaginal length and patency, and post-surgical dilation compliance is of the utmost importance to avoid future surgical procedures and complications [7].

Successful treatment has previously been classified into two categories: anatomical success and functional success. Anatomical success is largely defined as a vaginal canal of adequate length, typically at least 7 cm [17]. Functional success is considered more meaningful and is defined as the ability to achieve successful and satisfactory intercourse or the ability to accept the largest dilator without pain or discomfort [17-20]. Studies have found no correlation between the initial vaginal length and success of mechanical dilation, indicating that a vaginal dimple or less is sufficient with a motivated patient [18,20]. The frequency of dilation treatments seems to have a high influence, with one study reporting a greater change in vaginal length, a higher functional success rate, and a shorter duration of treatment for those patients that routinely completed their dilation therapy [17]. Although primary dilation has proven to be successful, surgery has been found to have a higher rate of functional success than dilation therapy [19].

Because of the potential surgical complications and difficulty of patient compliance, we proposed the use of a modification of our previously described intestinal expansion sleeve method to expand the vaginal canal length in a single step procedure in rats. We hypothesized that the vaginal expansion sleeve (VES) would induce lengthening of the vaginal canal without lesion to the vaginal wall through the process of ‘distraction vaginogenesis’, a derivative of distraction enterogenesis described in studies focused on treating short gut syndrome with longitudinal expansion devices that lengthen the bowel through distractive mechanical forces [21,22]. In the attempt to evaluate a novel self-expanding device as a potential treatment option for vaginal agenesis, this proof-of-concept study aims to create and mechanically characterize the VES device and to histologically evaluate the expansion of the vaginal wall following deployment.

### Materials and Methods

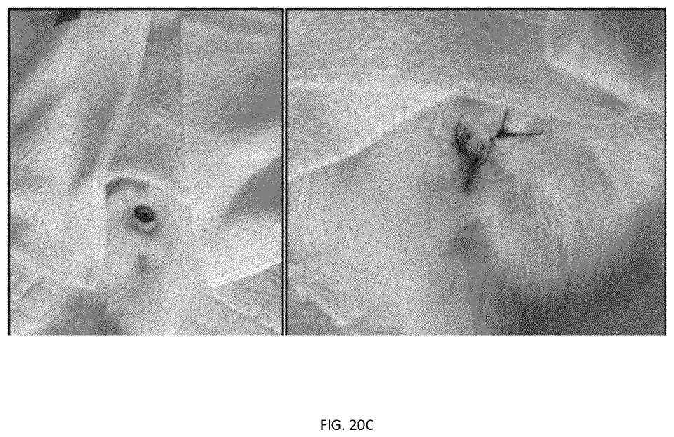

The VES is made of a woven 5 mm cylindrical layered polyethylene terephthalate with helicoid trusses characterized by isometric ends. Initially, each VES was cut to 20 mm in length. Caps comprised of the Biocompatible Photopolymer Resin Surgical Guide (Form-labs, Somerville, MA, USA) were secured onto each end using epoxy (FIG. 20A). However, the two caps interfered with the compression of the sleeve. After initial biomechanical testing, the VES was redesigned to have a coating of liquid rubber on each end to prevent fraying of the material, and a cap was kept only at the distal end to aid in suturing the device in place at the introitus. Additionally, barium was mixed into the Flex Seal® (Swift Response Inc.; Weston, FL, USA) to allow for post-operative visualization of the device.

Mechanical characterization of the VES was performed utilizing an Instron 8874 Biaxial Servo Hydraulic Fatigue Testing System. Cyclical compression and decompression of the VES device at a rate of 50 mm/min determined the force load (in newtons) that VES exerted at 10%, 20%, 30%, 40%, and 50% compression (FIG. 20B).

VES devices were implanted in the vaginal canal of deceased Sprague Dawley rats. The device was pre-contracted by diametric expansion before being deployed into the vaginal canal. Post-insertion, the VES was anchored and secured using three nonabsorbable 4-0 silk sutures at the exterior end of the vaginal canal (FIG. 20C). The vaginal canals were then harvested, post-expansion canal lengths were measured ex vivo, and tissue was prepared for preliminary histological analysis.

Vaginal canal tissues were fixed in 3.7% phosphate buffered formaldehyde for 24 h and then transferred to the LSU Pathology Core laboratory. Specimens were transferred through graded alcohol series and then into paraffin. Sections 5 μm in size were cut and processed, deparaffinized, and then stained for hematoxylin/eosin and mounted on slides. Photographs of specimens were taken, and the tissue layer thicknesses were evaluated using ImageJ (NIH, Bethesda, MD, USA, https://imagej.nih.gov/ij/) (accessed on 24 Jun. 2022). Statistical differences between treatment groups were analyzed using the two-tailed Students' t-test. A p-value of 0.05 or less was considered significant.

FIG. 20A shows the original VES design inserted into deceased rats. FIG. 20B shows Biomechanical characterization testing of original VES design. FIG. 20D shows VES post-Binsertion into the rat vaginal canal (left) and after suturing into place (right).

### Results

### Mechanical Characterization

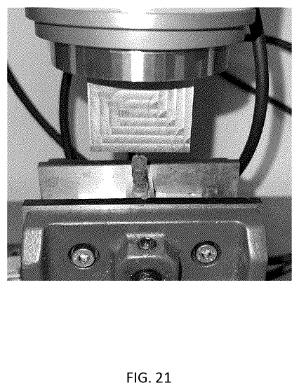

Testing of the biomechanical characterization of VES revealed that the caps on each end of the sleeve not only compromised the sleeve's compression but also restricted the sleeve from re-expanding to its original length after compression (FIG. 21). For this reason, further biomechanical testing was conducted using sleeves without caps on each end and with sleeves cut to 30 mm instead of 20 mm to compensate for the loss in cap length. The length of each device was measured pre- and post-compression with calipers to verify the post-compression expansion to the original length. The average nominal length of each VES was 30.13 mm, and the average post-compression length of each VES was 29.86 mm. The expansive force of each sleeve was recorded at 10%, 20%, 30%, 40%, and 50% compression

FIG. 21 shows failed re-expansion of double-capped VES post-compression.

TABLE 3
Average expansive force of VES at various compression intensities.

| Compression (%) | 10 | 20 | 30 | 40 | 50 |
|---|---|---|---|---|---|
| Force (N) | 0.34 ± 0.09 | 0.74 ± 0.05 | 1.16 ± 0.09 | 1.98 ± 0.11 | 11.69 ± 3.45 |

### Ex Vivo Insertion

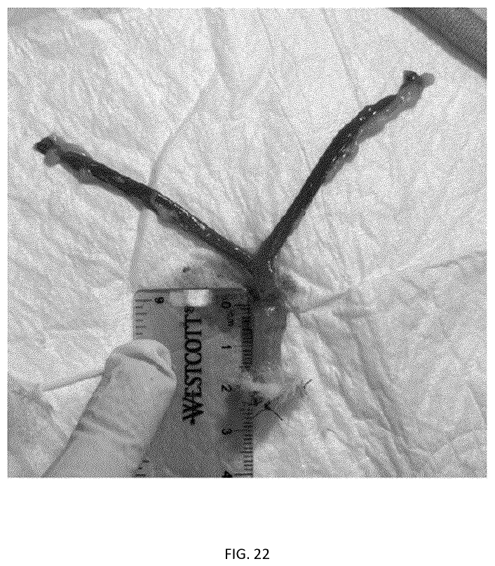

Seven VES devices were inserted into the vaginal canals of deceased Sprague Dawley rats and then the post-insertion vaginal lengths were immediately measured ex vivo (FIG. 22). The mean pre-insertion vaginal length was 18.29±3.35 mm, and the mean post-insertion vaginal length was 23.64±2.43 mm, resulting in a mean vaginal lengthening of 5.36±1.18 mm (Table 4). There was a 32.5±23.6% increase (p=0.004, Students t-test) in the vaginal length from pre- to post-insertion.

FIG. 22 shows harvested vaginal canal with the inserted VES.

TABLE 4
Vaginal canal lengths before VES insertion
and after VES insertion.

| | 1 | 2 | 3 | 4 | 5 | 6 | 7 |
|---|---|---|---|---|---|---|---|
| Pre-insertion (mm) | 13 | 16 | 16 | 22 | 21 | 21 | 19 |
| Post-insertion (mm) | 21.5 | 22 | 26 | 25 | 25 | 26 | 20 |
| Expansion (%) | 65.4 | 37.5 | 62.5 | 13.6 | 19.0 | 23.8 | 5.3 |

### Histological Analysis

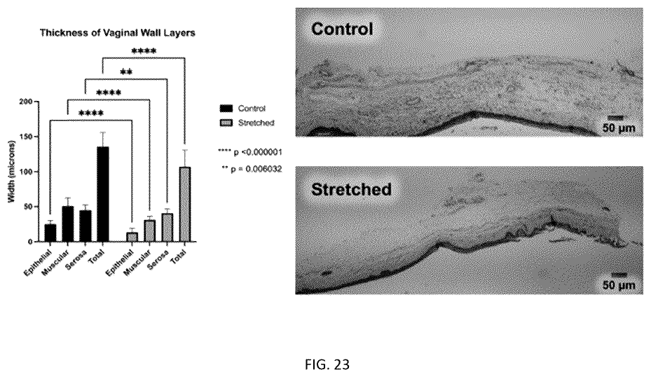

Histology demonstrated significant thinning across the epithelial, muscular, and serosal layers when compared to the control vaginal tissue samples, resulting in total vaginal wall thinning (FIG. 23 and Table 5) in all tissue layers.

TABLE 5
Vaginal tissue layer widths in the control and stretched tissues.

| Tissue Layer | Mean Control (μm) | Mean Stretched (μm) |
|---|---|---|
| Epithelial | 24.93 ± 5.30 | 13.17 ± 5.89 |
| Muscular | 50.70 ± 11.88 | 31.01 ± 5.07 |
| Serosa | 45.00 ± 7.45 | 40.29 ± 6.73 |
| Total | 135.76 ± 20.23 | 107.11 ± 23.76 |

FIG. 23. shows tissue layer thickness in the control and stretched vaginal tissue, assessed on the hematoxylin and eosin-stained slides.

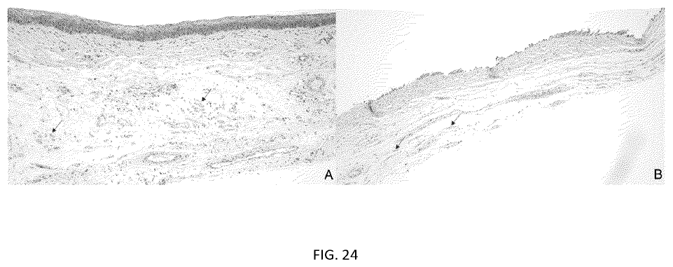

Review of the hematoxylin and eosin (H&E) stained slides revealed significant thinning of the overall vaginal wall as well as thinning of the individual histologic layers of the vaginal wall tissue following VES insertion. The control tissue showed organized, maturing squamous mucosa with a well-defined basal layer. The submucosa showed loose fibrous connective tissue with a zone of muscle fibers in loosely-formed bundles and blood vessels scattered throughout (FIG. 24A). The stretched tissue showed near-complete denudation of the squamous mucosa with an incomplete basal cell layer remaining attached to the un-derlying soft tissue. The depicted squamous mucosa is most representative of the stretched tissue, but occasional areas in some rats show more residual squamous mucosa, up to full thickness. The submucosa in the stretched tissue is thinner and more compressed than the control tissue, and the muscle fibers are elongated and nearly imperceptible at this magnification (FIG. 24B).

Black arrows show the muscle fibers in the control (FIG. 24A) and stretched (FIG. 24B) vaginal wall tissue. (FIG. 24A) Control tissue with unremarkable overall tissue width, organized, maturing squamous mucosa, and bundles of muscle fibers with some interspersed connective tissue (H&E, ×100). (FIG. 24B) Stretched tissue with reduced overall width, squamous disruption with only a partial basal layer remaining intact, and muscle fibers that are elongated and thinner than the control muscle fibers (H&E, ×100).

FIG. 24 shows histological data of muscle fibers in control and stretched vaginal wall tissue, under either an IES or VES embodiment.

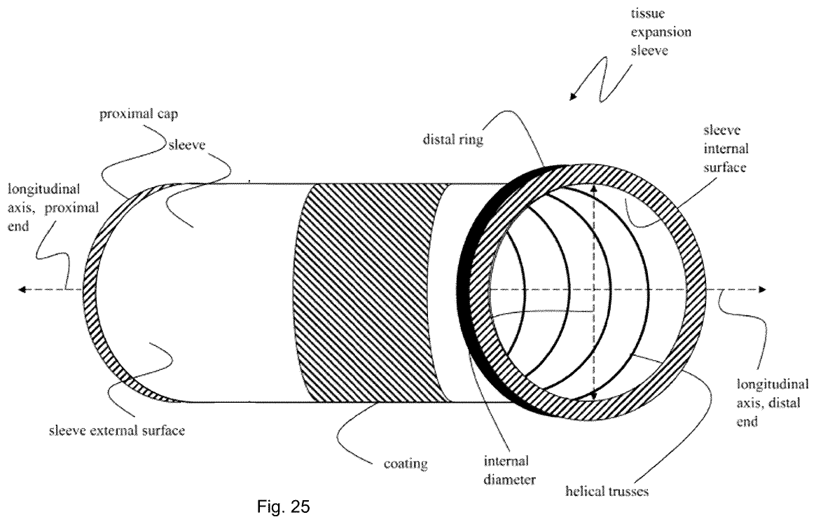

FIG. 25 shows a schematic view of an expansion sleeve, under either an IES or VES embodiment. Under an embodiment, the tissue expansion sleeve comprises a sleeve internal and external surface. The sleeve comprises a coating on its outer surface. Under an embodiment, the tissue expansion sleeve includes a distal end ring and/or a proximal end ring. The body of the sleeve comprises a woven material, wherein the woven material comprises helical trusses. The composition of the woven material may comprise a thermoplastic, wherein the thermoplastic is selected from the group consisting of polyethylene terephthalate (PET), polyethylene (PE), polypropylene (PP), polymethylmethacrylate (PMMA), polycarbonate (PC), polyvinyl chloride (PVC), polyetheretherketone (PEEK), or a combination thereof. The sleeve may be coated and crosslinked with polyvinyl alcohol which becomes a chemically reactive surface capable of binding amine-containing therapeutics such as biologics, hormones, and small biologic modifiers. One such therapeutic agent is GLP-2, which has vasodilatory, anti-inflammatory, and tissue regenerative properties.

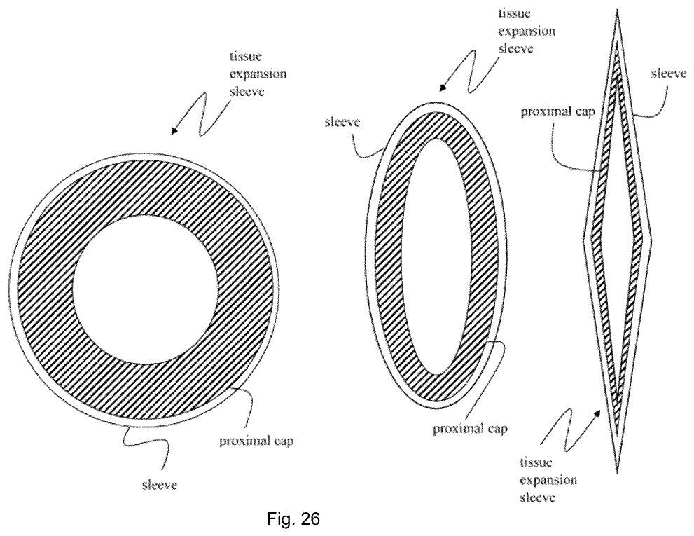

FIG. 26 shows components of an expansion sleeve, under either an IES or VES embodiment.

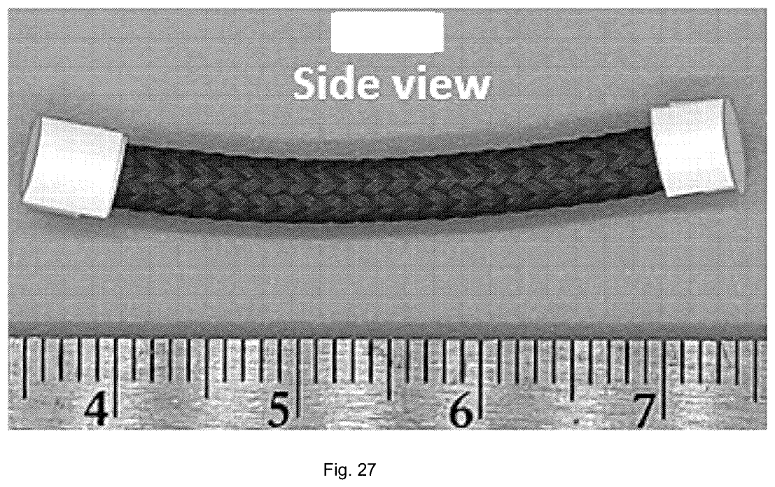

FIG. 27 shows an expansion sleeve, under either an IES or VES.

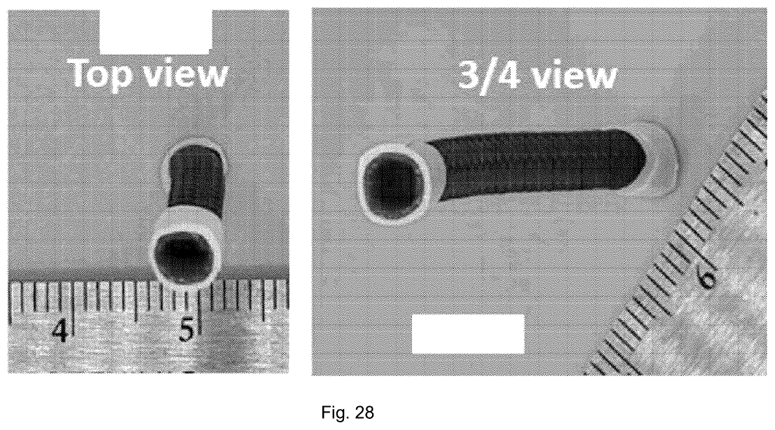

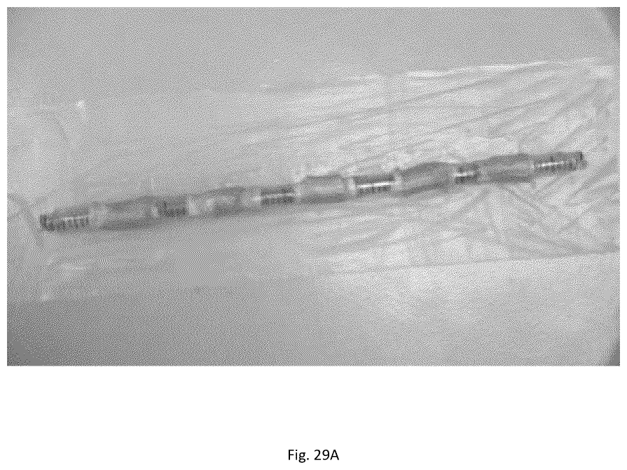

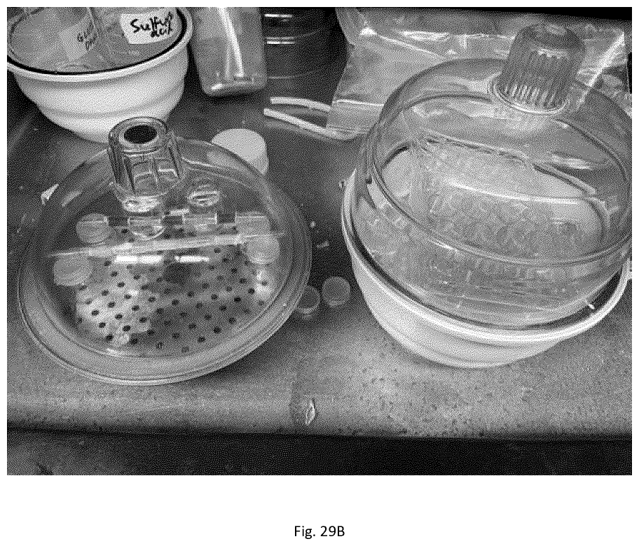

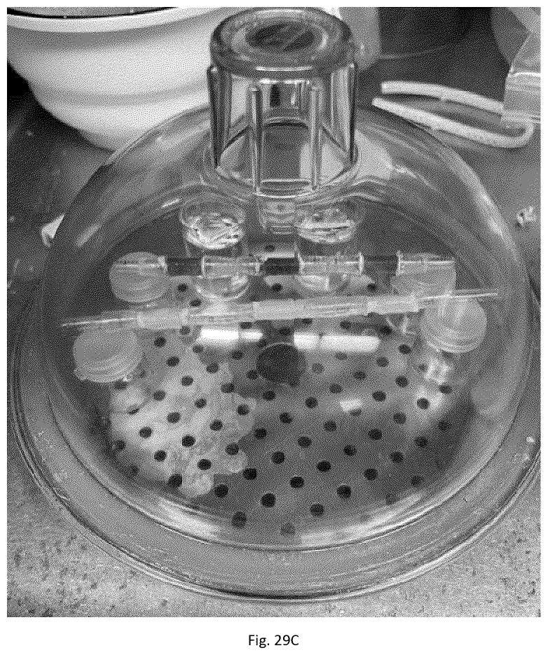

FIG. 28 shows an expansion sleeve, under either an IES or VES.

### Discussion

Vaginal atresia can lead to significant sexual dysfunction and therefore needs to be treated to attain successful penetrative intercourse. Treatment can lead to improved sexual satisfaction and quality of life [3]. The current treatment options for vaginal atresia have been proven to be successful, but each has its drawbacks. First-line treatment of serial dilation has promising results but relies heavily on patient compliance [2]. Additionally, surgical intervention has higher rates of serious complications and is typically reserved for those who have failed mechanical dilation [7]. In this study, we proposed an alternative therapy to mechanical dilation by utilizing a vaginal expansion sleeve (VES) that mechanically lengthens the vaginal canal in a single step, without relying on patient compliance. The VES may also have an additive role to either dilation therapy or a surgical option. For example, the VES could help to increase vaginal length and diameter in a patient who has not achieved functional vaginal success through other means.

Many patients that present with vaginal atresia have difficulty adjusting to their diagnosis emotionally. Successful creation of a neovagina can relieve a great deal of these psychological stresses, however, patients have reported that the process of dilation therapy serves as a constant reminder that they are ‘abnormal’ [4]. We anticipate that our device can lessen the frequency of this sentiment by performing the dilation therapy without manual effort or continuous attention from the patient. In this way, we hope the VES device can provide an easier path emotionally in the treatment of vaginal atresia. Theoretically, the desired vaginal canal can be achieved by customizing the VES to the patient's evolving or lengthening vagina. The VES could be 3D printed to exert slowly increasing force to achieve a goal. Multiple VES deployments may be necessary to achieve the optimal canal length. This method of vaginal lengthening could even be used to salvage a patient's vaginal canal who failed with other therapies.

Our proposed device is derived from another study focused on the use of distraction enterogenesis in the treatment of short gut syndrome through an intestinal expansion sleeve (IES) [21]. Distraction enterogenesis utilizes longitudinal expansion devices that inflict distractive mechanical force for an immediate increase in the small intestine length while maintaining tissue continuity and architecture [22]. We aim to repurpose the use of this IES device for ‘distraction vaginogenesis’ in the treatment of vaginal atresia. Placement of this device in a patient with a limited dimple on the perineum may require initial dilation therapy to develop a ‘landing zone’ for the VES. Thus, the device would be used in a case of severe vaginal atresia after a limited canal has been established to provide a space to deploy.

Testing of the biomechanical characterization revealed that compressing the sleeve to 50% of its original length yielded the highest exertional force load at 11.69±3.45 N. Compressing the VES device to only 40% of its original length resulted in a lower exertional force load at only 1.98±0.11 N, suggesting that the device contraction must be maintained while being inserted and anchored into the vaginal canal in order to achieve maximal lengthening. Anchoring the device to the external vaginal orifice was achieved using non-absorbable sutures to ensure the force exerted was directed inward toward the proximal end of the vagina. The use of absorbable sutures was considered, but once dissolved, the force exerted would be equalized on both ends of the device, causing less force to be directed toward lengthening as the device springs out bidirectionally and the device extrudes out of the vagina. Moreover, biomechanical testing revealed that the post-expansion sleeve length is nearly equivalent to the original pre-compression length. This demonstrates how we can approximate the length of the vaginal canal by adjusting the length of our device. Ex vivo insertion revealed an average of 32.5±23.6% in the immediate increase in vaginal length. However, whether the increased vaginal length is maintained or the vagina returns to its initial length once the device is removed requires further study in a live animal model. Furthermore, live animal trials are warranted to optimize the duration of implantation of the VES device required to elicit a more permanent stretch in the vaginal wall tissue.

Histologic analysis revealed an overall thinning of the vaginal wall tissue following VES insertion. The compression of the soft tissue and elongation of the muscle fibers indicate vaginal stretching and do not show significant connective tissue or muscle damage. However, the squamous mucosa disruption is significant in the stretched vaginal wall tissue. Because the rats in this study were dead at the time of the VES insertion, the vaginal wall tissue was not able to react in a physiological manner. Physical stretching of the vaginal wall tissue is possible, but this study could not address any of the other effects that may occur in the vaginal wall if this study was to be performed in live rats. The significant squamous mucosa changes seen in the stretched tissues in this study may not be seen in live rats, since live rats may exhibit a physiologic counterforce or other protective measures to ensure that the squamous mucosa integrity is maintained during and after the VES insertion. Chemical signaling, inflammatory response, and injury repair would be expected to occur in a live rat, and further studies are necessary to describe these effects. The enduring effects on the vaginal wall and potential permanent remodeling of the vagina following both short- and long-term VES insertion should be examined from a histologic standpoint to determine the optimal use of the VES to benefit patients with the least associated morbidity. There were several limitations of our study. The main limitation is that we used a rat model. The rat vagina typically has a length of around 15-20 mm and a diameter of around 3-5 mm, both of which are smaller than the 6-10 cm length and the 1.5-5 cm diameter typical of a normal human vagina [23]. However, it has been documented that there is an anatomical correspondence that would allow studies in the rat model to be extrapolated to humans [23,24]. In the current study, we measured a maximal force exercised by the VES device of 11.69±3.45 N. This is enough force to stretch the smaller rat vagina, but the force required of a human vagina would be larger, with one study by Cosson et al. finding a human vaginal tensile strength of 44.28±20.29 N [25]. Furthermore, in this study on the murine model, we documented an expansion of 5.36±1.18 mm, which is one order smaller than what has been documented for successful human vaginal lengthening [16]. Although this is a more modest result, reconfiguring the VES to allow greater expansion capability is anticipated, and the length, diameter, and exertional force of the proposed device would therefore need to be scaled to fit human vaginal standards. In light of the scalability needed, the results of our study need to be further corroborated with experiments on human tissues.

Our device requires some depth to the vagina in order to be inserted and may not be deployable in very shallow vaginal dimples. However, we may consider the use of the VES device as an adjunct to mechanical dilation therapy. Studies have found that shallow vaginal dimples are still amenable to traditional dilation therapy, and there is no correlation between the starting vaginal length and the success of dilation [18,20]. Moreover, one study following 16 women through dilation treatment reported that the frequency of dilation therapy significantly decreased to only once a week after 1-3 months of treatment, citing factors such as time and effort constraints, privacy issues, and a lowered perceived vaginal lengthening as reasons to reduce therapy frequency [26]. The reduced frequency in therapy resulted in significantly smaller vaginal lengths than those patients who dilated frequently. Our device could eliminate these negative factors as it does not require the patient to manually perform the treatment. As soon as the patient achieves a minimal length, the VES device can be implanted. Time commitments and issues finding privacy to manually conduct dilation and decreased motivation from a perceived stalled lengthening would essentially be eliminated. The likelihood of a larger end vaginal length would increase, and the need for surgery may be avoided.

Another limitation is that this study was performed on a normal rat vagina and not an atretic one. A vaginal canal with significant atresia will likely be more challenging to elongate. Our initial biomechanical tests were performed on tissue derived from deceased adult rats with fully formed vaginal canals. Hypoplastic vaginal canals may have a different tissue composition of epithelial, smooth muscle, and even substantial scar tissue from prior attempts at treatments that may alter the performance of the VES [12]. Additional effects from VES deployment on vaginal neuronal and vascular supplies would need further study in a live animal model over a longer period of time. Furthermore, we did not obtain full compression of the VES device due to the size of the resin cap. This could be resolved by using an absorbable cap on the proximal end that dissolves after VES anchoring. We will implement this change in future in vivo studies focused on the tissue remodeling over time. Immediate vaginal elongation was successfully achieved with the VES in deceased Sprague Dawley rats by thinning the vaginal canal tissue without compromising it. VES expansion to its equilibrium dimension maintained longitudinal vaginal canal tension, which may permit remodeling and increased canal length while preserving vascular and nervous supplies. Further studies are needed in vivo to address and verify this theory of ‘distraction vaginogenesis’.

### Conclusions

VES is a unique approach for potentially achieving vaginal canal elongation in women with vaginal atresia and represents a promising alternative or adjunct to primary dilation by eliminating vigilant patient compliance. Devices creating distraction vaginogenesis could even be used to improve a surgically created vaginal canal. The VES device is less invasive than traditional surgical interventions, suggesting that this device represents a viable alternative to mechanical dilation and surgical interventions. However, our results still require further in vivo studies for biocompatibility and mechanical characterization, in order to more fully understand and validate this novel approach.

Future research on the VES will include the determination of the in vivo elongation of the vaginal canal in Sprague Dawley rats. This method of elongation of the vaginal canal may have other patient populations that could benefit such as transgender. The VES could be impregnated with estrogen, VEGF, or GLP-2 to enhance the mucosal stimulation and improve the architecture of the neovagina. Studies will need to be performed to determine if these ‘enhanced’ expansion sleeves have benefits. A VES with dissolvable cross links may be better tolerated in future patients. These cross linked devices would exert force in a slower measured time frame. Further investigation is warranted in order to optimize the duration of implantation and the forced expansion necessary to facilitate ‘distraction vaginogenesis’.

### REFERENCES

1. Nakhal, R. S.; Creighton, S. M. Management of vaginal agenesis. J. Pediatr. Adolesc. Gynecol. 2012, 25, 352-357.
2. Oelschlager, A. M.; Debiec, K.; Appelbaum, H. Primary vaginal dilation for vaginal agenesis: Strategies to anticipate challenges and optimize outcomes. Curr. Opin. Obstet. Gynecol. 2019, 28, 345-349.
3. Kang, J.; Chen, N.; Song, S.; Zhang, Y.; Ma, C.; Ma, Y.; Zhu, L. Sexual function and quality of life after the creation of a neovagina in women with Mayer-Rokitansky-Küster-Hauser syndrome: Comparison of vaginal dilation and surgical procedures. Fertil. Steril. 2020, 113, 1024-1031.
4. Edmonds, D. K.; Rose, G. L.; Lipton, M. G.; Quek, J. Mayer-Rokitansky-Küster-Hauser syndrome: A review of 245 consecutive cases managed by a multidisciplinary approach with vaginal dilators. Fertil. Steril. 2012, 97, 686-690.
5. Gulía, C.; Baldassarra, S.; Zangari, A.; Briganti, V.; Gigli, S.; Gaffi, M.; Signore, F.; Vallone, C.; Nucciotti, R.; Costantini, F. M.; et al. Androgen insensitivity syndrome. Eur. Rev. Med. Pharmacol. Sci. 2018, 22, 3873-3887.
6. Minto, C. L.; Liao, K. L.; Conway, G. S.; Creighton, S. M. Sexual function in women with complete androgen insensitivity syndrome. Fertil. Steril. 2003, 80, 157-164.
7. ACOG Committee Opinion No. 728 Summary: Müllerian Agenesis: Diagnosis, Management, and Treatment. Obstet. Gynecol. 2018, 131, 196-197.
8. Frank, R. The formation of an artificial vagina without operation. Am. J. Obstet. Gynecol. 1938, 35, 1053-1055.
9. Willemsen, W. N.; Kluivers, K. B. Long-term results of vaginal construction with the use of Frank dilation and a peritoneal graft (Davydov procedure) in patients with Mayer-Rokitansky-Küster syndrome. Fertil. Steril. 2015, 103, 220-227.
10. Apfel, V. R.; Takano, C. C.; Marquini, G. V.; de Jarmy di Bella, Z.; Girão, M.; Sartori, M. Treatment for vaginal agenesis: A prospective and comparative study between vaginal dilation and surgical neovaginoplasty. Int. J. Gynaecol. Obstet. Off. Organ Int. Fed. Gynaecol. Obstet. 2021, 157, 574-581.
11. Routh, J. C.; Laufer, M. R.; Cannon, G. M., Jr.; Diamond, D. A.; Gargollo, P. C. Management strategies for Mayer-Rokitansky-Kuster-Hauser related vaginal agenesis: A cost-effectiveness analysis. J. Urol. 2010, 184, 2116-2121.
12. Guarino, N.; Scommegna, S.; Majore, S.; Rapone, A. M.; Ungaro, L.; Morrone, A.; Grammatico, P.; Marrocco, G. A. Vaginoplasty for disorders of sex development. Front. Endocrinol. 2013, 4, 29.
13. Cheikhelard, A.; Bidet, M.; Baptiste, A.; Viaud, M.; Fagot, C.; Khen-Dunlop, N.; Louis-Sylvestre, C.; Sarnacki, S.; Touraine, P.; Elie, C.; et al. Surgery is not superior to dilation for the management of vaginal agenesis in Mayer-Rokitansky-Küster-Hauser syndrome: A multicenter comparative observational study in 131 patients. Am. J. Obstet. Gynecol. 2018, 219, 281.e1-281.e9.
14. Ozkan, O.; Erman Akar, M.; Ozkan, O.; Dog an, N. U. Reconstruction of vaginal agenesis. Ann. Plast. Surg. 2011, 66, 673-678.
15. Callens, N.; De Cuypere, G.; De Sutter, P.; Monstrey, S.; Weyers, S.; Hoebeke, P.; Cools, M. An update on surgical and non-surgical treatments for vaginal hypoplasia. Hum. Reprod. Update 2014, 20, 775-801.
16. Callens, N.; De Cuypere, G.; Wolffenbuttel, K. P.; Beerendonk, C. C.; van der Zwan, Y. G.; van den Berg, M.; Monstrey, S.; Van Kuyk, M. E.; De Sutter, P.; Belgian-Dutch Study Group on DSD; et al. Long-term psychosexual and anatomical outcome after vaginal dilation or vaginoplasty: A comparative study. J. Sex. Med. 2012, 9, 1842-1851.
17. Gargollo, P. C.; Cannon, G. M., Jr.; Diamond, D. A.; Thomas, P.; Burke, V.; Laufer, M. R. Should progressive perineal dilation be considered first line therapy for vaginal agenesis? J. Urol. 2009, 182, 1882-1891.
18. Roberts, C. P.; Haber, M. J.; Rock, J. A. Vaginal creation for müllerian agenesis. Am. J. Obstet. Gynecol. 2001, 185, 1349-1353.
19. Nadarajah, S.; Quek, J.; Rose, G. L.; Edmonds, D. K. Sexual function in women treated with dilators for vaginal agenesis. J. Pediatr. Adolesc. Gynecol. 2005, 18, 39-42.
20. Rock, J. A.; Reeves, L. A.; Retto, H.; Baramki, T. A.; Zacur, H. A.; Jones, H. W., Jr. Success following vaginal creation for Müllerian agenesis. Fertil. Steril. 1983, 39, 809-813.
21. Clayton, S.; Alexander, J. S.; Solitro, G.; White, L.; Villalba, S.; Winder, E.; Boudreaux, M.; Veerareddy, P.; Dong, E.; Minagar, A.; et al. Self-expanding intestinal expansion sleeves (IES) for short gut syndrome. Pediatr. Surg. Int. 2022, 38, 75-81.
22. Koga, H.; Sun, X.; Yang, H.; Nose, K.; Somara, S.; Bitar, K. N.; Owyang, C.; Okawada, M.; Teitelbaum, D. H. Distraction-induced intestinal enterogenesis: Preservation of intestinal function and lengthening after reimplantation into normal jejunum. Ann. Surg. 2012, 255, 302-310.
23. McCracken, J. M.; Calderon, G. A.; Robinson, A. J.; Sullivan, C. N.; Cosgriff-Hernandez, E.; Hakim, J. C. Animal models and alternatives in vaginal research: A comparative review. Reprod. Sci. 2021, 28, 1759-1773.
24. Moalli, P. A.; Howden, N. S.; Lowder, J. L.; Navarro, J.; Debes, K. M.; Abramowitch, S. D.; Woo, S. L. A rat model to study the structural properties of the vagina and its supportive tissues. Am. J. Obstet. Gynecol. 2005, 192, 80-88.
25. Cosson, M.; Lambaudie, E.; Boukerrou, M.; Lobry, P.; Crépin, G.; Ego, A. A biomechanical study of the strength of vaginal tissues: Results on 16 post-menopausal patients presenting with genital prolapse. Eur. J. Obstet. Gynecol. Reprod. Biol. 2004, 112, 201-205.
26. Callens, N.; Weyers, S.; Monstrey, S.; Stockman, S.; van Hoorde, B.; van Hoecke, E.; Cools, M.; Cuypere, G. D.; Hoebeke, P. Vaginal dilation treatment in women with vaginal hypoplasia: A prospective one-year follow-up study. Am. J. Obstet. Gynecol. 2014, 211, 228.e1-228.e12.

### Example 6

The use of protein-based therapeutics is limited by their route of administration and the inability to confine therapeutics to their site of action. One innovative approach can chemically bind therapeutics to medical devices, allowing localized and concentrated delivery of therapeutics to the site of action. This study aims to evaluate if GLP-2 can be covalently bound to our Vaginal Expansion Sleeve (VES) and Intestinal Expansion Sleeve (IES) in measurable quantities.

### Methods

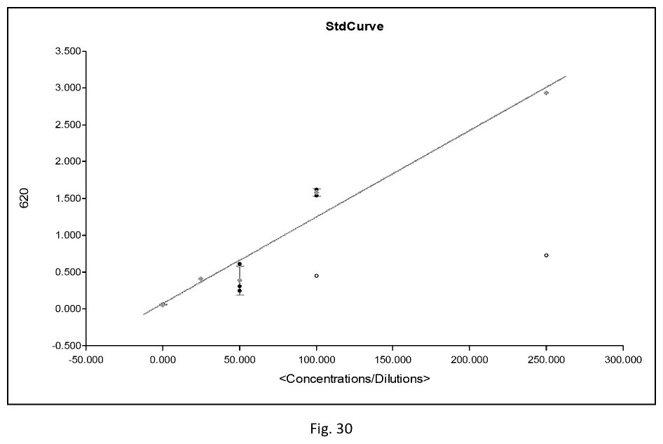

Expansion sleeves were coated and crosslinked with polyvinyl alcohol (PVA) making a chemically reactive surface capable of binding amine-containing therapeutics such as GLP-2. A standard curve was created by adding 250 ug, 100 μg, 50 ug, 25 ug, and 0 ug of GLP-2 into respective wells (FIG. 30). A rabbit anti-GLP-2 antibody followed by a goat anti-rabbit IgG alkaline phosphatase secondary antibody was added to the wells to allow the addition of SeraCare KPL BluePhos Microwell Phosphate Substrate System. Once added, the color would change from yellow to blue depending on the concentration of GLP-2 bound antibodies, allowing the absorbance to be read at 620 nm to generate the standard curve and calculate the concentration of GLP-2 on the PVA-coated sleeves.

### Results

Addition of 50 ug of GLP-2, each IES and VES each device bound an average of 22.69±9.32 ug/cm² of GLP-2 after adjustment for an external surface area of 9.425 cm², allowing for 44% of added GLP-2 to remain fixated to the PVA coated sleeves.

### Conclusion

Current GLP-2 dosing in humans in 0.6 mg/70 kg. With an external surface area of 9.425 cm², each sleeve is capable of giving a localized delivery of 213.85 ug of GLP-2, which is 25.2× greater dose than systemic dosing. This methodology makes it possible to add dramatically lower doses of therapeutic agents to get the same effect as systemic administration of the GLP-2 drug while also avoiding systemic effects.

### Example 7

### Purpose

The use of protein-based therapeutics is limited by their route of administration and the inability to confine therapeutics to their intended site of action. Several approaches have been advanced such as the incorporation of these agents in dissolvable time-release matrices or repeated administrations. One innovative approach would be to chemically bind therapeutics to medical devices covalently, allowing localized and concentrated delivery of therapeutics to the site of action. This study aims to evaluate if GLP-2 can be covalently bound to our Vaginal Expansion Sleeve (VES) and Intestinal Expansion Sleeve (IES) in measurable quantities.

### Methods

Here we have coated and crosslinked expansion sleeves with polyvinyl alcohol which becomes a chemically reactive surface capable of binding amine-containing therapeutics such as biologics, hormones, and small biologic modifiers. One such therapeutic agent is GLP-2, which has vasodilatory, anti-inflammatory, and tissue regenerative properties. If appropriately targeted, GLP-2 administration would have a more long-lasting effect at the site of interest and less systemic effects.

### Results

This study evaluated whether and to what extent GLP-2 can be covalently bound to an implantable medical device to achieve localized delivery at a concentration supporting therapeutic levels. Each IES and VES had an external surface area of 9.425 cm² and we were able to bind an average of 22.69±9.32 ug/cm² onto each device. Each well on the 24-well plate has a surface area of 1.93 cm² giving the standard curve concentrations 125.95 ug/cm², 66.38 ug/cm², 23.77 ug/cm², 14.58 ug/cm², and 0 ug/cm² for the 250 ug, 100 μg, 50 ug, 25 ug, and 0 ug added. Overall, this method allows for 44% of the added GLP-2 to be bound to the PVA coating.

### Conclusion

GLP-2 was found in significant quantities on the device, which can enhance its action at the site of vaginal or intestinal lengthening. Current GLP-2 dosing in humans in 0.6 mg/70 Kg which corresponds to 8.57 ug/Kg. Described herein, we can bind GLP-2 at 22.69±9.32 ug/cm² on the sleeves. Since the external surface area is 9.425 cm², this gives the capacity for a localized delivery of 213.85 ug of GLP-2, this is a 25.2× greater dose than systemic dosing. This methodology makes it possible to add dramatically lower doses of therapeutic agents to get the same effect as systemic administration of the GLP-2 drug while also avoiding systemic effects.

### Introduction

The efficacy of therapeutics, including biologics (antibodies, cytokines, hormones, etc.) and small molecule drugs, is limited by biodistribution, off target effects, and cost prohibition. Therapeutics are often delivered systemically. However, by limiting the delivery to the site of action (i.e., local delivery), treatment outcomes can be improved, and side effects can be mitigated. Described herein are compositions and methods that maintain continuous, local delivery of therapeutics thereby avoiding the unwanted effects of systemic delivery. This method can be used, for example, in treating tumors. In some embodiments, the compositions and methods described herein comprise expansion sleeves coated and/or crosslinked with a reactive surface to bind amine-containing therapeutics. In certain embodiments, the expansion sleeves can be coated and/or crosslinked with polyvinyl alcohol (PVA) using a reaction with glutaraldehyde and sulfuric acid.

### Methods

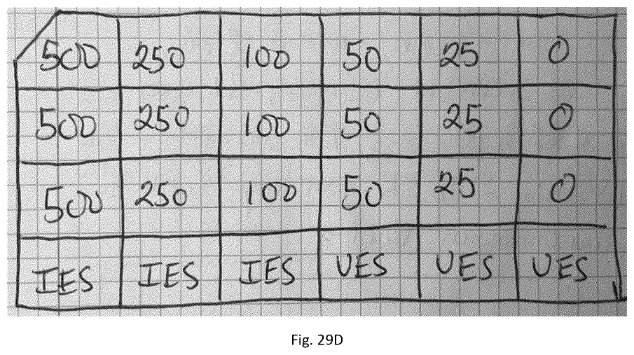

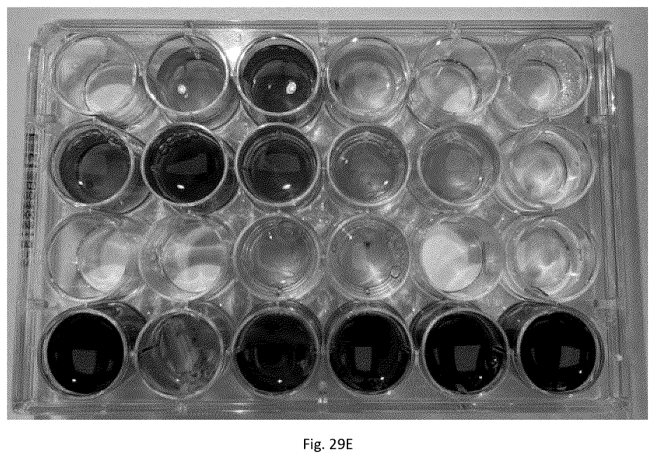

Intestinal expansion sleeves and vaginal expansion sleeves were precontracted over a glass pipette to allow us to paint them with 5% Polyvinyl alcohol (PVA) (FIG. 29A). After painting a coat of PVA, the sleeves were allowed to dry and another coat was added for a total of 5 PVA coats on each sleeve. After the 5th coat dried, the precontracted sleeves were put into a 150 mm Nalgene desiccator to allow them to crosslink (FIG. 29B and FIG. 29C). Crosslinking was done by adding separate open beakers of 3 mL of 25% Glutaraldehyde and sulfuric acid so that the vapors could react inside the chamber to crosslink the PVA onto the sleeves. After being in the chamber for 48 hours the sleeves were taken out of the chamber and 50 μL of a 1 mg/mL GLP-2 solution was pipetted carefully onto each sleeve. This was allowed to dry for 30 minutes before wrapping each sleeve with parafilm and placing into the fridge overnight. The next day we removed the sleeves and placed them each into their labeled well on the following 24-well plate. To make the standard curve, a small layer of 5% PVA was painted onto the bottom of the intended-to-use wells of a 24-well plate. 6 wells were left blank to account for the 6 wells needed for the sleeves. The PVA on the 24-well plate was allowed to dry and then crosslinked as stated above. The concentration gradient (FIG. 29D) was made by adding 500 uL, 250 uL, 100 uL, 50 uL, 25 uL, and 0 uL of 1 mg/mL GLP-2 into their labeled wells creating the 24-well plate with 3 wells for each concentration (250 ug, 100 μg, 50 ug, 25 ug, and 0 ug) and 3 wells for each type of sleeve (IES and VES).

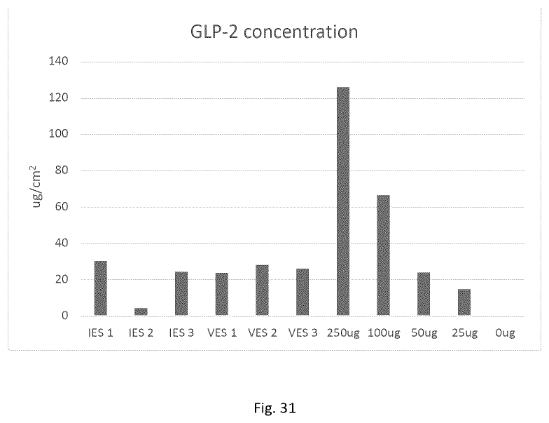

After the respective GLP-2 concentrations was added to the wells and the drug coated sleeves were put in their wells, the plate was put in the fridge overnight. A blocking solution of 1 mg/mL of milk powder in Millipore water was adding to block the plate for 1 hour followed by 3 washes of PBS for 5 minutes each wash. A rabbit GLP-2 antibody solution was made by adding 40 μL of rabbit GLP-2 antibody was added into 50 mL of Millipore water. 2 mL of the rabbit GLP-2 antibody solution was added to each well on the 24-well plate then put back in the fridge overnight. After incubation, another 3× wash for PBS was completed before adding 2 mL of secondary antibody solution of Anti-rabbit IgG alkaline phosphatase produced in goat to each well. The secondary antibody solution was made by 20 mL of antibody into 40 mL of Millipore water. This was allowed to bind for 2 hours before washing off with another 3×PBS wash. Next the SeraCare KPL BluePhos Microwell Phosphate Substrate System was added to each well for 20 minutes to allow the color reaction to happen. After the color reaction was completed with the yellow solution turning blue (FIG. 29E), the plate was read at an absorbance of 620 wavelength to generate the standard curve (FIG. 30) and calculate the concentration of GLP-2 on the PVA coated sleeves (FIG. 31).

### Results

Our intestinal expansion sleeves and vaginal expansion sleeves were painted with polyvinyl alcohol, crosslinked, and covalently bonded with GLP-2. Each device had an external surface area of 9.425 cm2 and we were able to bind an average of 22.69±9.32 ug/cm2 onto each device.

The 24-well plate had a surface area of 1.93 cm2 giving the concentrations of 125.95, 66.38, 23.77, 14.58, 0.00 ug/cm² for the 250 ug, 100 μg, 50 ug, 25 ug, and 0 ug wells. The average GLP-2 bound to the device was 22.69±9.32 ug/cm2

After adding 50 ug of GLP-2 onto each sleeve, the proven amount of GLP-2 bound to the device was 22.69±9.32 ug/cm2. This corresponds to the 23.7 ug/cm2 for the 50 ug well of the standard curve. By adding 50 ug of GLP-2 to these sleeves we have a 45% added drug stayed bound to the device after being washed multiple times.

### Example 8

Short bowel syndrome (SBS) is characterized by insufficient intestinal length leading to malabsorption and malnutrition. The bowel adapts to SBS via intestinal dilation and delayed gastric emptying, but still often requires long term parenteral nutrition. Surgical options to lengthen the bowel pose significant risks and often provide limited expansion. Distraction enterogenesis has been proposed as a technique to induce intestinal lengthening for SBS. Here we experimentally evaluated an intestinal expansion sleeve (IES) for lengthening the bowel. We hypothesized that deployment of the IES device will result in significant intestinal lengthening in vivo.

A Roux-en-Y in the jejunum of 7 rats was created for isolated IES deployment. The IES was precontracted over Bucatini noodle and inserted into the isolated roux limb. After 4 weeks of deployment, the rats were sacrificed and the Roux-en-Y length was recorded, harvested, and stained for histological analysis. A paired t-test was performed to compare initial and final roux limb lengths.

Intestinal distraction was evaluated at 4 weeks post deployment of the IES, resulting in a significant increase in roux limb length from an average of 43.6±14.4 mm to 56.4±20.8 mm (p=. 043, n=7) providing a 30.2% elongation. IES samples showed variation in mucosal and submucosal integrity as well as varying bowel wall thickness in response to IES lengthening. In samples with partial mucosal erosion, the basal/regenerative layers of the mucosa were preserved.

Distraction enterogenesis with significant intestinal lengthening in vivo has been achieved with the IES device. Histologic changes suggest all bowel functional layers and attributes are maintained through distraction enterogenesis. Future constructs of the IES may benefit from the addition of immunomodulators. Increasing intestinal mass with these devices may change the treatment paradigm for SBS.

### Introduction

Short bowel syndrome (SBS) is characterized by insufficient intestinal length leading to malabsorption and malnutrition. Partial surgical resection of the small bowel is the most common cause of SBS, but it can also be caused by any disease or injury that prevents proper function of the bowel (1,2). Acquired SBS is more common than congenital forms (3). In pediatrics, SBS is most commonly due to necrotizing enterocolitis, malrotation with volvulus, or intestinal malformations including omphalocele or gastroschisis (2,4,5).

The overall incidence of pediatric SBS was 22.1 affected per 1,000 admissions to the neonatal intensive care unit (NICU), and 24.5 affected per 100,000 live births (6). Normal adult small intestine length is around 600 cm (19.7 ft) and colon length is 150 cm (4.9 ft) for a total GI tract length of 750 cm (approximately 25 ft); however, in SBS patients, the average GI tract length is only 6.5 ft (3). Clinical symptoms arise when the remaining gastrointestinal tract is unable to compensate for the lost length, therefore SBS is largely based on the loss of functional absorptive tissue (7). SBS symptoms vary depending on how much and which parts of the bowel are missing, but diarrhea is common causing dehydration, weight loss, and malnutrition (8). Malnourished patients have swelling of the abdomen, muscle wasting, and vitamin/mineral deficiencies (including vitamins K, B1 and B12 as well as potassium, calcium, magnesium, and zinc) causing an array of symptoms (3,8).

Less invasive management for SBS includes long-term total parenteral nutrition (TPN) support, and pharmacological management helping to improve intestinal function and/or slow the transit time through the intestines (1,4,9). SBS patients adapt naturally by delayed gastric emptying and intestinal dilation, but this can take years and often requires parenteral nutrition for adequate growth (10). Surgical options to increase bowel length have been performed mostly in the pediatric population as opposed to adults, and these procedures may pose significant risks and often provide limited expansion (7). These methods range from intestinal transplants to various surgical elongation procedures such as the Bianchi procedure and serial transverse enteroplasty (STEP) procedure (7,11-13). The Bianchi procedure, otherwise known as the longitudinal intestinal lengthening and tailoring (LILT) procedure, consists of longitudinally dividing the bowel in half, then stapling the bowel (5,11). After making the two sections of bowel with a smaller diameter, the ends are sewn together, doubling the length of bowel (2,11). In order for the Bianchi procedure to be successful, the initial bowel needs a minimal length of 20-40 cm and the diameter must be dilated to at least 200% (2). The STEP procedure is performed by stapling the bowel transversely across half of the luminal diameter and alternating sides, lengthening the bowel and decreasing the diameter (1,12). However, an important aspect to consider with these procedures is that it can take years to dilate the bowel to a diameter required in both of these instances (14). The earlier the intestinal elongation procedures are performed, the less TPN is needed for these patients (4).

Distraction-induced enterogenesis, a form of controlled tissue expansion, has been shown to produce significant bowel lengthening with proliferation of the epithelial structure allowing preservation of intestinal function (15-18). This was done in an excised length of bowel that was re-implanted back into the rat model, showing that the re-implanted bowel still grows and functions like native bowel (15). Spencer et al. used a hydraulic-driven dual concentric piston made with telescoping syringes that were inserted into the lumen of the isolated bowel in pigs. After device stabilization, the pigs were closed up to allow for one week of recovery before device activation to elongate the bowel, resulting in significantly longer bowel segments compared to the control segments. These lengthened segments had an almost 2-fold increase in surface area (19). In rats, Safford et al. used an intestinal lengthening device that was inserted into the lumen of the bowel and secured to the external oblique fascia. After recovery, the device was manually lengthened by 1 mm per day. Once the device was removed, the intestine was still 114% longer in length and had increased function (20).

We performed an ex vivo investigation using small intestines from New Zealand White rabbits, where our IES device showed a 36.2±11% increase in small bowel length, however, we also noted intestinal wall thinning (10). Similarly, our previous ex vivo mechanical testing proof of concept study in Sprague Dawley rat small intestines showed that the force required to rupture the small bowel was 1.88±0.21 N, while the IES device exerted 0.22±0.01 N, only 11.7% of the tension which produced failure. Deployment of the IES device ex vivo resulted in a 21±8% increase in rat small bowel length. However, as before, this produced mucosal loss and tissue thinning (21). Our studies show that our IES device is capable of lengthening the intestinal tissue without failure, (i.e. providing longitudinal expansion while preserving the intestinal architecture.) In vivo, the ‘distractive’ forces of the IES device should allow the small bowel mucosa to accommodate and remodel while stretching, thereby lengthening the bowel while avoiding persistent thinning of the intestinal walls. IES devices could be used to treat both pediatric patients and those with acquired SBS by restoring small bowel length through ‘distraction enterogenesis’. Our hypothesis is that deployed IES devices result in significant intestinal lengthening in vivo while preserving normal intestinal architecture.

### Methods

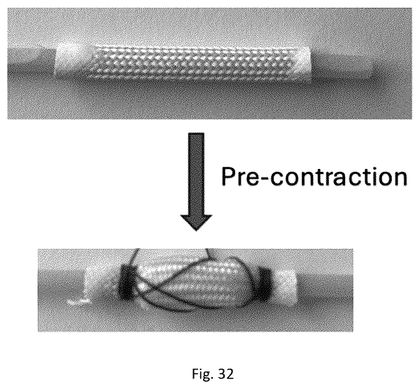

The Intestinal Expansion Sleeve (IES) is a 30 mm cylindrical material with helicoid trusses. Each end is painted with a rubber sealant to prevent fraying of the device as it is precontracted over a rigid polymer support. Once the device is precontracted, it is fixated using absorbable suture to allow the device to be deployed into the intestine in a pre-contracted state and re-expand once the absorbable suture dissolves (FIG. 32).

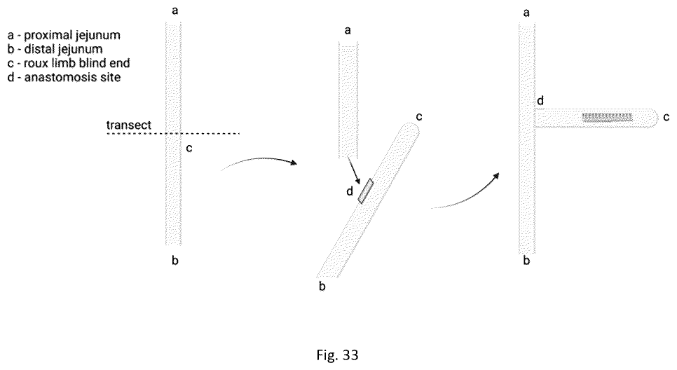

In Sprague-Dawley rats, a 5 cm Roux-En-Y was constructed with 5-0 silk. (FIG. 33). After creation of the roux limb, the precontracted IES device is inserted into the isolated intestinal limb and fixated using 4-0 silk permanent suture at the ends. The absorbable suture holding the device in a precontracted state and the polymer support dissolves allowing the device to re-expand. As the device re-expands, the permanent suture holds it in place in the small bowel, and the force promotes ‘distraction enterogenesis’.

Control rats also underwent surgery in two separate ways. One group of controls had surgery to create a Roux-En-Y of the small bowel, but no IES device was inserted. The other control group had a Roux-En-Y performed and a non-contracted IES device inserted and stabilized. All rat groups were allowed 4 weeks of daily observation and weighing before final measurement of the roux limb. The roux limb and anastomosis were isolate, resected, and placed in 3.7% phosphate-buffered formaldehyde for 24 hours, and processed by LSU Pathology Core laboratory. Following fixation and processing, tissue was embedded in paraffin for sectioning. 5 μm thick sections were cut and stained with hematoxylin/eosin for analysis.

FIG. 32 shows precontraction and compression of an IES device.

FIG. 33 shows creation of the Roux-En-Y.

### Results

### Intestinal Lengthening

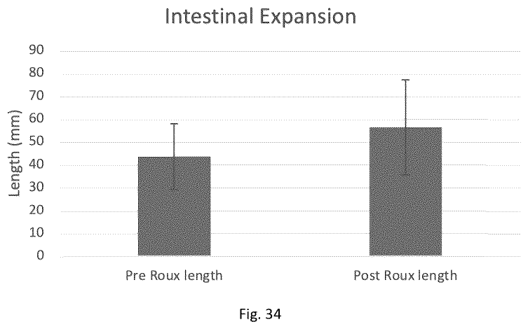

FIG. 34 shows Intestinal Length Pre and Post Expansion (deployment of IES device in rat small intestines over one month produced lengthening of 43.6±14.4 mm to 56.4±20.8 mm (p=. 043, n=7), a 30.2% elongation).

The 2 control rats with roux limbs with no IES device the roux length was initially 40 mm and this remained at 40 mm; similarly, the 2 controls with roux limbs with unexpanded IES devices had an initial roux length of 60 mm which remained at 60 mm of length.

TABLE 6
Intestinal Length Pre and Post Expansion

| | Pre Roux | Post Roux | Roux limb | Compressed | IES | Initial | Final |
|---|---|---|---|---|---|---|---|
| | length | length | growth | IES length | length | weight | weight |
| | (mm) | (mm) | (%) | (mm) | (mm) | (g) | (g) |
| IES 1 | 35 | 46 | 31.43 | 15 | 30 | 741 | 781 |
| IES 2 | 20 | 34 | 70 | 20 | 30 | 659 | 652 |
| IES 3 | 40 | 39 | −2.5 | 20 | 30 | 557 | 566 |
| IES 4 | 40 | 53 | 32.5 | 20 | 30 | 731 | 741 |
| IES 5 | 60 | 58 | −3.33 | 15 | 30 | 590 | 593 |
| IES 6 | 60 | 95 | 58.33 | 17 | 30 | 617 | 610 |
| IES 7 | 50 | 70 | 40 | 19 | 30 | 788 | 780 |

Histologic analysis of the non-contracted and pre-contracted IES-exposed intestinal tissue revealed predominantly intact surface mucosa and villous architecture with foci of partially eroded surface mucosa, while the control Roux-En-Y no IES intestinal tissue architecture was completely preserved and unremarkable. Of note, suture material was identified in multiple sections of both control and IES-exposed intestinal tissue, consistent with post-operative surgical changes. Distinct suture granulomas with varying degrees of surrounding foreign body granulomatous inflammatory reaction are seen in both control and IES-exposed tissue. Additionally, the bowel wall of IES-exposed tissue contains mild scattered chronic inflammation, independent of the suture granulomas. The bowel wall thickness was variable among control and IES-exposed tissue, with no distinct trend in thickness based on the presence or absence of an IES device.

### Discussion

Short Bowel Syndrome affects 24.5 out of every 100,000 live births and has diverse symptoms reflecting on the degree of shortening and anatomical location of the bowel which is affected (3,6). Multiple treatment options exist for SBS ranging from intestinal adaptation with TPN support to surgical management (4). Approaches which improve intestinal adaptation (remodeling) increase bowel diameter and gastroparesis, allowing slower transit time for nutrient absorption (2,13,22). However, such adaptation can take years to occur and is a goal of most surgical procedures for SBS (14,19).

Our previous ex vivo study demonstrated that IES devices provide adequate lengthening with a distractive force well below the failure load of the rat small bowel and is safe to use in vivo without occurrences of small bowel rupture/perforation (21). In this in vivo study, we successfully showed that deployment of our IES device can significantly lengthen the small bowel, allowing ‘distraction enterogenesis’ to take place which preserves normal intestinal architecture following the lengthening procedure.

After a one-month deployment of our IES device in rats, the average intestinal length increased from 43.6±14.4 mm to 56.4±20.8 mm (p=. 043, n=7), a 30.2% elongation. Currently, we are testing the IES device in a blind-ended roux limb, out of line with the intestinal flow, requiring an invasive surgery. However, we anticipate an in-line deployment of the IES device to allow insertion of the device without a need to operate. As IESs are contracted, the diameter increases allowing for better fixation to the intestinal walls. When they re-expand, the diameter correspondingly decreases. Our goal is to ultimately be able to deploy the precontracted IES device by a long feeding tube, allowing delivery and fixation to the intestinal wall. Therefore, as the device re-expands to lengthen the intestine, it will decrease in diameter, release itself from the intestinal wall, and be passed out through the gastrointestinal canal. This offers the possibility of an intermediate management when TPN and intestinal adaptation are not enough, while avoiding invasive surgeries like intestinal transplants, Bianchi procedure, and STEP procedure that require general anesthesia and hospitalization. Additionally, treatment with the IES device can be done before gradual intestinal adaptation occurs, unlike the Bianchi procedure and STEP procedures (11,12).

Histologic review reveals normal small bowel histology with preserved surface mucosa and villous architecture. Although there are some foci of partial mucosal erosion in both groups of IES-exposed tissue, this appears to be likely secondary to physical contact with the IES device. Notably, the basal portion of the mucosa is preserved, therefore, the small bowel does retain its ability for self-repair following removal of the device. Suture material, distinct submucosal suture granulomas, and associated submucosal granulomatous reaction were observed, but are not significantly different when compared to control tissues with IES exposure. Given the final lengths following stretching with the IES device, the suture and associated inflammatory responses do not seem to compromise the ability of the bowel wall to lengthen. The chronic inflammation and apparent thinning seen in the bowel wall away from the granulomatous suture reaction is likely secondary to bowel wall stretching and does not appear to irreversibly affect the tissue structure. The histologic findings are encouraging and suggest that the use of the IES device does not cause severe or permanent damage to the bowel wall. Therefore, bowel structure and function of the bowel are maintained following IES device exposure.

We recognize that the lack of post-treatment evaluation of expanded intestinal function as a limitation of this study. We cannot state if new intestinal growth will be retained long term and function as well as normal intestine. Histologically, in places with mucosal erosion, the basal/regenerative layer remains intact, therefore, we believe that the mucosa can self-repair and retain normal function. However, we cannot currently confirm this. Longer observation times would be needed to allow for the mucosa to regenerate to determine if the superficial mucosal layers and bowel wall thickness would return to normal. Another limitation of the study is that we only deployed a single IES device per procedure. Each SBS patient requires different distraction lengths to become independent of TPN. A study done by Dubrovsky et al. describes a required bowel length (cm) to body weight (kg) ratio of 1.0 cm/kg is necessary for adult SBS patients to become independent of TPN (23). In future studies, multiple rounds of IES deployment will likely be needed to develop adequate lengthening to treat SBS. We anticipate that several IES could be deployed simultaneously or that multiple rounds of IES deployment could achieve sufficient lengthening to treat SBS.

In this study the initial Roux-En-Y lengths were not all equal, ranging from lengths of 20 mm to 60 mm. This made the standard deviation of the pre-IES and post-IES deployment lengths larger than what we anticipated. In future studies, we will have Roux-En-Y lengths set to a fixed standard to better determine the lengthening capacity of the 3 cm IES device. Also, measurement of the Roux-Ex-Y lengths is subject to human error, which largely reflects two of the seven roux limbs having a decrease in length post-IES lengthening. Due to the nature of this device, a decrease in length is not expected unless a measurement error occurs or failure of IES device to deploy. Using this rat model, we are unable to fully gauge discomfort of the IES device. However, as all animals maintained their weight and daily activity with no agitation during handling, we can conclude that they do not display overt pain or distress (24).

### Conclusion

This study shows that significant gut lengthening can be achieved through distraction enterogenesis using our novel IES device. Histopathological analysis displayed no differences between the controls and IES device. Future testing of multiple IES devices, and drug impregnated IES devices should determine when and how IES can be applied as important treatments for patients with SBS.

### REFERENCES

1. Muto, M., Kaji, T., Onishi, S., Yano, K., Yamada, W., & Ieiri, S. (2022). An overview of the current management of short-bowel syndrome in pediatric patients. Surgery Today, 1-10.
2. Muff, J. L., Sokolovski, F., Walsh-Korb, Z., Choudhury, R. A., Dunn, J. C., Holland-Cunz, S. G., & Vuille-dit-Bille, R. N. (2022). Surgical treatment of short bowel syndrome—the past, the present and the future, a descriptive review of the literature. Children, 9 (7), 1024.
3. NORD. Short Bowel Syndrome—NORD (National Organization for Rare Disorders). NORD (National Organization for Rare Disorders). Published 2019. https://rarediseases.org/rare-diseases/short-bowel-syndrome/
4. Guillen B, Atherton N S. Short Bowel Syndrome. [Updated 2023 Jul. 17]. In: StatPearls [Internet]. Treasure Island (FL): StatPearls Publishing; 2024 January-. Available from: https://www.ncbi.nlm.nih.gov/books/NBK536935/
5. Höllwarth, M. E. (2017). Surgical strategies in short bowel syndrome. Pediatric surgery international, 33, 413-419.
6. Puttonen M, Tuominen S, Ukkola-Vuoti L, Lassenius M I, Virtanen H, Merras-Salmio L, Pakarinen M P. Pediatric Short Bowel Syndrome: Real-World Evidence on Incidence and Hospital Resource Use From a Finnish Data Lake. J Pediatr Gastroenterol Nutr. 2023 Oct. 1; 77 (4): 479-485. doi: 10.1097/MPG.0000000000003894. Epub 2023 Jul. 25. PMID: 37490606; PMCID: PMC10501354.
7. Massironi, S., Cavalcoli, F., Rausa, E., Invernizzi, P., Braga, M., & Vecchi, M. (2020). Understanding short bowel syndrome: Current status and future perspectives. Digestive and Liver Disease, 52 (3), 253-261.
8. Billiauws, L., Maggiori, L., Joly, F., & Panis, Y. (2018). Medical and surgical management of short bowel syndrome. Journal of Visceral Surgery, 155 (4), 283-291.
9. Seetharam, P., & Rodrigues, G. (2011). Short bowel syndrome: a review of management options. Saudi Journal of Gastroenterology, 17 (4), 229-235.
10. Clayton S, Alexander J S, Solitro G, White L, Villalba S, Winder E, Boudreaux M, Veerareddy P, Dong E, Minagar A, Dao H N, Sorrells D. Self-expanding intestinal expansion sleeves (IES) for short gut syndrome. Pediatr Surg Int. 2022 January; 38 (1): 75-81. doi: 10.1007/s00383-021-05024-8. Epub 2021 Oct. 28. PMID: 34709433.
11. Bianchi A (1980) Intestinal loop lengthening—a technique for increasing small intestinal length. J Pediatr Surg 15:145-151
12. Kim H B et al (2003) Serial transverse enteroplasty (STEP): a novel bowel lengthening procedure. J Pediatr Surg 38:425-429
13. Millar, A. J. (2013). Non-transplant surgery for short bowel syndrome. Pediatric Surgery International, 29, 983-987.
14. Tappenden, K. A. (2014). Intestinal adaptation following resection. Journal of Parenteral and Enteral Nutrition, 38, 23S-31S.
15. Koga H, Sun X, Yang H, Nose K, Somara S, Bitar K N, Owyang C, Okawada M, Teitelbaum D H. Distraction-induced intestinal enterogenesis: preservation of intestinal function and lengthening after reimplantation into normal jejunum. Ann Surg. 2012 February; 255 (2): 302-10. doi: 10.1097/SLA.0b013e318233097c. PMID: 21997804; PMCID: PMC3259228.
16. Iyer, K. R. (2014). Surgical management of short bowel syndrome. Journal of Parenteral and Enteral Nutrition, 38, 53S-59S.
17. Hosseini, H. S., Taylor, J. S., Wood, L. S., & Dunn, J. C. (2020). Biomechanics of small intestine during distraction enterogenesis with an intraluminal spring. Journal of the Mechanical Behavior of Biomedical Materials, 101, 103413.
18. Ralls, M. W., Sueyoshi, R., Herman, R. S., Utter, B., Czarnocki, I., Si, N., . . . & Teitelbaum, D. H. (2013). Mesenteric neovascularization with distraction-induced intestinal growth: enterogenesis. Pediatric surgery international, 29, 33-39.
19. Spencer A U, Sun X, El-Sawaf M, et al. Enterogenesis in a clinically feasible model of mechanical small-bowel lengthening. Surgery. 2006; 140 (2): 212-220. doi: 10.1016/j.surg.2006.03.005
20. Safford S D, Freemerman A J, Safford K M, Bentley R, Skinner M A. Longitudinal mechanical tension induces growth in the small bowel of juvenile rats. Gut. 2005; 54 (8): 1085. doi: 10.1136/gut.2004.061481
21. O'Quin, C., Clayton, S. D., Trosclair, L., Meyer, H., Dao, N. H., Minagar, A., . . . & Sorrells, D. (2024). Distraction Enterogenesis in Rats: A Novel Approach for the Treatment of Short Bowel Syndrome. Pathophysiology, 31 (3), 388-397.
22. Pironi, L. (2016). Definitions of intestinal failure and the short bowel syndrome. Best practice & research Clinical gastroenterology, 30 (2), 173-185.
23. Dubrovsky G, Huynh N, Thomas A L, Shekherdimian S, Dunn J C. Double plication for spring-mediated in-continuity intestinal lengthening in a porcine model. Surg (United States). 2019; 165 (2). doi: 10.1016/j.surg.2018.07.028.
24. Carstens, Gary P. Moberg, Recognizing Pain and Distress in Laboratory Animals, ILAR Journal, Volume 41, Issue 2, 2000, Pages 62-71, https://doi.org/10.1093/ilar.41.2.62

A device is described herein comprising an expansion sleeve comprising a cylindrical woven material, wherein the cylindrical woven material comprises helical trusses, wherein an outer surface of the cylindrical woven material is coated with a biologically active material.

In embodiments, longitudinal compression of the expansion sleeve causes axial expansion.

In embodiments, axial contraction of the expansion sleeve causes longitudinal lengthening of the expansion sleeve.

In embodiments, the expansion sleeve is configured for insertion into a tissue canal structure.

In embodiments, the tissue canal structure comprises a Roux limb of a Roux-en-Y procedure.

In embodiments, the tissue canal structure comprises a continuous intestinal section.

In embodiments, the tissue canal structure comprises a vaginal canal.

In embodiments, the insertion comprises pre-compressing the expansion sleeve.

In embodiments, pre-compressing the expansion sleeve comprises compressing the sleeve on a dilator.

In embodiments, the dilator comprises a rapidly degrading organic material.

In embodiments, insertion comprises anchoring the pre-compressed expansion sleeve within the tissue canal structure.

In embodiments, the anchoring comprises affixing a first end of the pre-compressed expansion sleeve to the tissue canal structure using a first plurality of sutures.

In embodiments, the anchoring comprises affixing a second end of the pre-compressed expansion sleeve to the tissue canal structure using a second plurality of sutures.

In embodiments, the anchored pre-compressed expansion sleeve decompresses over a duration of time.

In embodiments, the decompression causes axial contraction of the expansion sleeve.

In embodiments, the decompression causes longitudinal lengthening of the expansion sleeve.

In embodiments, the longitudinal lengthening of the expansion sleeve lengthens the tissue canal structure.

In embodiments, helical trusses comprise opposing helical threads.

In embodiments, the opposing helical threads comprise a mesh weave.

In embodiments, the cylindrical woven material comprises a metal material, a polymer material, or a composite material.

In embodiments, the polymer material comprises a thermoplastic.

In embodiments, the thermoplastic is selected from the group consisting of polyethylene terephthalate (PET), polyethylene (PE), polypropylene (PP), polymethylmethacrylate (PMMA), polycarbonate (PC), polyvinyl chloride (PVC), polyetheretherketone (PEEK), or a combination thereof.

In embodiments, the bioactive material comprises a polyvinyl alcohol (PVA) coating reacted with a dialdehyde, wherein the PVA reacted with the dialdehyde is covalently linked to an amine-containing therapeutic.

In embodiments, the dialdehyde is selected from the group consisting of glutaraldehyde (GA), propanedial, butanedial, hexanedial, heptanedial, octanedial, nonanedial, decandial, or a dialdehyde containing more than 10 carbons.

In embodiments, the amine-containing therapeutic comprises a biologic or a small molecule.

In embodiments, the biologic comprises a peptide, a protein, a growth factor, a cell, a peptide hormone, an antibody, a steroid, or any combination thereof.

In embodiments, the small molecule comprises an antibiotic, an enzyme inhibitor, a receptor modulator, an ion channel modulator, an antiviral, anti-cancer drug, a hormone modulator, a neurotransmitter modulator, iodine, bromine, or a divalent cation.

In embodiments, the peptide hormone comprises GLP-2.

In embodiments, the amine-containing therapeutic comprises immunosuppressive activity, immunomodulating activity, angiogenic activity, or any combination thereof.

1. A device comprising,
an expansion sleeve comprising a cylindrical woven material, wherein the cylindrical woven material comprises helical trusses, wherein an outer surface of the cylindrical woven material is coated with a biologically active material.

2. The device of claim 1, wherein longitudinal compression of the expansion sleeve causes axial expansion.

3. The device of claim 2, wherein axial contraction of the expansion sleeve causes longitudinal lengthening of the expansion sleeve.

4. The device of claim 3, wherein the expansion sleeve is configured for insertion into a tissue canal structure.

5. The device of claim 4, wherein the tissue canal structure comprises a Roux limb of a Roux-en-Y procedure.

6. The device of claim 4, wherein the tissue canal structure comprises a continuous intestinal section.

7. The device of claim 4, wherein the tissue canal structure comprises a vaginal canal.

8. The device of claim 4, where the insertion comprises pre-compressing the expansion sleeve.

9. The device of claim 8, wherein pre-compressing the expansion sleeve comprises compressing the sleeve on a dilator.

10. The device of claim 9, wherein the dilator comprises a rapidly degrading organic material.

11. The device of claim 8, wherein insertion comprises anchoring the pre-compressed expansion sleeve within the tissue canal structure.

12. The device of claim 11, wherein the anchoring comprises affixing a first end of the pre-compressed expansion sleeve to the tissue canal structure using a first plurality of sutures.

13. The device of claim 12, wherein the anchoring comprises affixing a second end of the pre-compressed expansion sleeve to the tissue canal structure using a second plurality of sutures.

14. The device of claim 11, wherein the anchored pre-compressed expansion sleeve decompresses over a duration of time.

15. The device of claim 14, wherein the decompression causes axial contraction of the expansion sleeve.

16. The device of claim 15, wherein the decompression causes longitudinal lengthening of the expansion sleeve.

17. The device of claim 16, wherein the wherein the longitudinal lengthening of the expansion sleeve lengthens the tissue canal structure.

18. The device of claim 1, wherein helical trusses comprise opposing helical threads.

19. The device of claim 18, wherein the opposing helical threads comprise a mesh weave.

20. The device of claim 1, wherein the cylindrical woven material comprises a metal material, a polymer material, or a composite material.

21. The device of claim 20, wherein the polymer material comprises a thermoplastic.

22. The device of claim 20, wherein the thermoplastic is selected from the group consisting of polyethylene terephthalate (PET), polyethylene (PE), polypropylene (PP), polymethylmethacrylate (PMMA), polycarbonate (PC), polyvinyl chloride (PVC), polyetheretherketone (PEEK), or a combination thereof.

23. The device of claim 1, wherein the bioactive material comprises a polyvinyl alcohol (PVA) coating reacted with a dialdehyde, wherein the PVA reacted with the dialdehyde is covalently linked to an amine-containing therapeutic.

24. The device of claim 23, wherein the dialdehyde is selected from the group consisting of glutaraldehyde (GA), propanedial, butanedial, hexanedial, heptanedial, octanedial, nonanedial, decandial, or a dialdehyde containing more than 10 carbons.

25. The device of claim 23, wherein the amine-containing therapeutic comprises a biologic or a small molecule.

26. The device of claim 25, wherein the biologic comprises a peptide, a protein,, a growth factor, a cell, a peptide hormone, an antibody, a steroid, or any combination thereof.

27. The device of claim 25, wherein the small molecule comprises an antibiotic, an enzyme inhibitor, a receptor modulator, an ion channel modulator, an antiviral, anti-cancer drug, a hormone modulator, a neurotransmitter modulator, iodine, bromine, or a divalent cation.

28. The device of claim 26, wherein the peptide hormone comprises GLP-2.

29. The device of claim 23, wherein the amine-containing therapeutic comprises immunosuppressive activity, immunomodulating activity, angiogenic activity, or any combination thereof.
